(19) United States
(12) Patent Application Publication
(10) Pub. No.: US 2015/0291951 A1
(43) Pub. Date: Oct. 15, 2015

(54) GENOME EDITING

(71) Applicant: MAYO FOUNDATION FOR MEDICAL EDUCATION AND RESEARCH, Rochester, MN (US)

(72) Inventors: Stephen C. Ekker, Rochester, MN (US); Jarryd M. Campbell, Rochester, MN (US); Victoria M. Bedell, Rochester, MN (US)

(21) Appl. No.: 14/410,279

(22) PCT Filed: Mar. 14, 2013
(86) PCT No.: PCT/US2013/031754

§ 371 Date: Dec. 22, 2014

Related U.S. Application Data
(60) Provisional application No. 61/663,451, filed on Jun. 22, 2012.
(60) Provisional application No. 61/701,540, filed on Sep. 14, 2012.

(51) Int. Cl. C12N 15/10

(52) U.S. Cl. CPC C12N 15/102

(57) ABSTRACT

This document provides materials and methods for editing a genome (e.g., an animal genome in vivo). For example, methods and materials for using a targeted endonuclease and a donor nucleic acid having a length within a particular range (e.g., from 20 to 95 nucleotides) to edit a genome are provided.

### CROSS-REFERENCE TO RELATED APPLICATIONS

This application claims benefit of priority from U.S. Provisional Application No. 61/701,540, filed on Sep. 14, 2012, and U.S. Provisional Application No. 61/663,451, filed on Jun. 22, 2012.

### STATEMENT AS TO FEDERALLY SPONSORED RESEARCH

This invention was made with government support under GM063904, DK084567, and DK083219, awarded by the National Institutes of Health. The government has certain rights in the invention.

### TECHNICAL FIELD

This disclosure relates to materials and methods for editing a genome (e.g., an animal genome in vivo). The methods and materials can involve using a targeted endonuclease and a donor nucleic acid having a length within a particular range (e.g., from 20 to 95 nucleotides).

### BACKGROUND

The zebrafish (Danio rerio) can be considered a premier teleostean model system. With strong biological and genomic similarities to other vertebrates, this organism is increasingly being used to study human biology and disease using a rich array of available in vivo genetic and molecular tools.

### SUMMARY

The ability to edit genomes (e.g., to edit genomes precisely) can be considered a bottleneck in life science, particularly for direct in vivo editing within model systems. This disclosure provides a transcription activator-like effector (TALE) nuclease toolbox that enables a new approach to in vivo genome editing using a non-mammalian vertebrate, the zebrafish (D. rerio). As described herein, TALE nucleases and donor nucleic acid can be used to perform homologous recombination successfully in species such as zebrafish. For example, this document demonstrates the ability to introduce genetic changes precisely at a TALE nuclease cut site in vivo using single-stranded DNA oligonucleotides as a donor sequence. Such methods can be used to introduce changes (e.g., small changes) at a TALE nuclease cut site in zebrafish. In some cases, the methods and materials provided herein can be used to introduce loxP-related sequences at two different TALE nuclease cut sites, thereby allowing for conditional allele generation in zebrafish and other model systems.

In addition, as described herein, particular scaffold backbones can be used in a manner that greatly increases the efficacy of artificial custom restriction endonucleases, TALE nucleases. For example, four of five (80%) +63 scaffold TALE nucleases exhibited DNA targeting rates that were higher than other tested TALE nucleases in zebrafish. Also, three of five (60%) +63 scaffold TALE nucleases exhibited bi-allelic conversion in somatic tissues, which can facilitate direct functional genomic analyses in injected animals (such as was previously accomplished using morpholino oligonucleotide knockdowns). This improved efficacy that yields bi-allelic conversion demonstrates that the TALE nucleases provided herein can be used in zebrafish and other systems (e.g., in vitro applications such as a single-step modifying approaches for iPSCs or gene therapy approaches).

In general, one aspect of this document features a method for modifying the genetic material of an organism. The method comprises, or consists essentially of, introducing into a cell of the organism: (i) a first nucleic acid encoding a first transcription activator-like effector (TALE) nuclease monomer, (ii) a second nucleic acid encoding a second TALE nuclease monomer, and (iii) a donor nucleic acid, wherein each of the first and second TALE nuclease monomers comprises a plurality of TAL effector repeat sequences and a FokI endonuclease domain, wherein the first TAL effector endonuclease monomer comprises the ability to bind to a first half-site sequence of a target DNA within the cell and comprises the ability to cleave the target DNA when the second TAL effector endonuclease monomer is bound to a second half-site sequence of the target DNA, wherein the target DNA comprises the first half-site sequence and the second half-site sequence separated by a spacer sequence, wherein the first and second half-sites have the same nucleotide sequence or different nucleotide sequences, and wherein the donor nucleic acid is from 20 to 85 (e.g., from 35 to 55) nucleotides in length and comprises a sequence that is heterologous to the target DNA and that is flanked by sequences that are similar or identical to the endogenous target nucleotide sequence. The organism can be a zebrafish embryo. The donor nucleic acid can be a single stranded DNA. The donor nucleic acid can be 40 to 50 nucleotides in length. The first TALE nuclease monomer, the second TALE nuclease monomer, or both the first and second TALE nuclease monomers can have a +63 scaffold backbone as set forth in SEQ ID NO:104.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a diagram illustrating the difference between the +231 (also referred to as TALE nuclease +231 or pTAL) and +63 TALE nuclease (also referred to as TALE nuclease +63 or GoldyTALEN) scaffold. Both scaffolds contain a SV40 nuclear localization (NLS) signal at the N-terminus and a FokI nuclease domain at the C-terminus. All numbers are relative to the DNA binding domain with −1 at the N-terminus and +1 at the C-terminus. The GoldyTALEN scaffold was made by truncating the pTAL scaffold to 152 amino acids at the N-terminus and 63 amino acids at the C-terminus. Numerous, smaller changes were highlighted in an amino acid comparison (FIG. 9). The highly active pT3TS transcription factor vector was used for mRNA synthesis. FIG. 7 includes the amino acid sequence of the +63 second-generation scaffold.

FIG. 1B is a schematic showing the layout of TALE nuclease target sites. TALE nucleases were targeted to exons with a spacer region that contained a restriction enzyme site for easy screening through the introduction of a restriction fragment length polymorphism (RFLP). Primers were asymmetrically placed around the targeted exon.

FIG. 1C is a series of pictures a gels indicating the relative activity of the +63 and +231 scaffolds as compared at two different loci, ponzr1 (top panel) and crhr1 (bottom panel). Each lane is a PCR-based DNA analysis of a single larva. Under each lane is the percent undigested DNA for that embryo, illustrating the increased activity of the +63 TALE nuclease scaffold.

FIG. 1D is a graph plotting TALE nuclease activity, and showing that ponzr1 TALE nucleases demonstrated a significant (p<10⁻¹⁶), 6-fold increase in activity with the +63 scaffold, and a significant (p<10⁻⁹), 15-fold increase in RFLP introduction at the crhr1 locus.

FIG. 1E is a picture of a gel from a cell-free restriction enzyme digestion assay, showing that ponzr1 TALE nucleases in the +63 scaffold were more active than TALE nucleases in the +231 scaffold. ponzr1 DNA is labeled in both uncut and cut forms.

FIG. 1F is a sequence comparison of RFLP changes induced by the +231 and +63 scaffolds, which did not demonstrate a significant difference in the types of insertion/deletions (indels) introduced at the ponzr1 locus. The crhr1 TALE nuclease yielded similar indels. The RFLP sites detected in somatic tissues were similar to those found in four germline mutations at the ponzr1 locus.

FIG. 2A is a series of pictures of gels showing amplified products from embryos injected with +63 TALE nucleases designed against the moesina (left panel), ppp1ca (middle panel) and cdh5 (right panel) genes. The spacer region contained a restriction enzyme site. Injection of +63 TALE nuclease mRNAs demonstrated nearly complete loss of the restriction enzyme site cutting in the amplicons of somatic tissue, suggesting bi-allelic gene targeting for each gene. Each lane is the amplification product from a group of 10 embryos. Mutant seq % refers to the percentage of amplicons that carry mutant sequences, as determined by sequencing approximately 10 clones.

FIG. 2B is a series of pictures of embryos injected with +63 TALE nucleases against cdh5, which phenocopied the morphant phenotype (Wang et al., Development, 137:3119-3128, 2010). Brightfield images (left panels) show pronounced cardiac edema in both +63 TALE nuclease- (middle) and MO-injected (bottom) larvae at 3 days post fertilization (dpf), as compared to controls (top). Vascular structure was visualized using the Tg(fli1-EGFP)^{y1} line (center panels). Normal trunk vascular patterning was observed in both TALE nuclease- (middle) and MO-injected (bottom) larvae. The Tg(gata1:dsRed) line (right panels) revealed greatly reduced circulation in larvae injected with either +63 TALE nucleases (middle) or MOs (bottom) as compared with controls (top).

FIG. 2C is a series of pictures of gels showing germline transmission of targeted disruptions using moesina (top), ppp1ca (middle) and cdh5 (bottom) +63 TALE nucleases. F1 embryos were derived from an outcross of F0 +63 TALE nuclease-injected founders, and genomic DNA was isolated in groups of 10 embryos. The amplicons showed resistance to restriction endonuclease digestion, indicating that the indels seen in somatic tissue were passed through the germline. Beneath each lane is the percent uncut, which estimates the percent of targeted alleles.

FIG. 2D shows the sequences of amplicons from DNA isolated from two individual embryos at each locus, confirming efficient germline transmission of mutant alleles.

FIG. 3A is a diagram showing single-stranded DNA (ssDNA) with sequence homology at the ponzr1 test site, which were used to introduce exogenous sequences into the genome in vivo.

FIG. 3B is a picture of a representative gel showing integration of an artificial EcoRV at the ponzr1 locus. Each lane is a PCR-based DNA analysis of a single zebrafish larva, and the cut bands demonstrate an EcoRV sequence insertion.

FIG. 3C is a sequence analysis of the three germline-transmitting lines. The first fish transmitting homology directed repair (HDR)-based genome changes through the germline (#1) yielded 7 out of 96 embryos with an incorporated EcoRV site. The genomes of all 7 embryos exhibited the same modified sequence. The second founder fish (#2) yielded 7 out of 46 embryos with EcoRV incorporation. All 7 embryos exhibited precise HDR-based addition of the EcoRV sequence. The third fish with germline transmission (#3) yielded 5 out of 18 embryos with an incorporated EcoRV site, and exhibited a mosaic germline as demonstrated by offspring with three different modified sequences. One embryo included precise HDR-based EcoRV addition. The other 4 embryos contained sequence insertions on the 5′ end with two embryos each harboring the specific sequences changes.

FIG. 3D is a picture of a representative gel showing mLoxP integration at the ponzr1 locus. Each lane is a PCR-based DNA analysis of a single zebrafish larva, and the PCR bands using primers against the mLoxP sequence demonstrate sequence insertion. Sequence analysis confirms the introduction of the engineered mLoxP sequence editing event.

FIG. 3E is a sequence analysis confirming that mLoxP insertion was seen in a second locus, crhr2. Three out of 10 sequences demonstrated HR, while seven of the 10 had mloxP insertion with indels on the 5′ end.

FIG. 4 is a diagram depicting the three classes of major outcomes caused by TALE nuclease-catalyzed double-stranded breaks in chromosomal DNA. First, error-prone NHEJ can produce an indel in and near the spacer region of the TALE nuclease binding site (bottom left). If a complementary ssDNA oligonucleotide is added in addition to the TALE nuclease, two different outcomes are noted. In the first, homologous recombination can precisely use the exogenous sequence information in the ssDNA to add sequence at the cut site (bottom middle). Alternatively, the ssDNA can act as a primer for 3′ integration of the oligonucleotide but the 5′ end may undergo error-prone NHEJ (bottom right).

FIG. 5A shows sequence confirmation of bi-allelic gene targeting in F0 +63 TALE nuclease-injected embryos. +63 TALE nucleases were designed against the sequences shown for the moesina, cdh5 and ppp1ca genes. In all cases the spacer region contained a restriction enzyme site. The amplification product from a group of 10 embryos was cloned, and individual clones were sequenced to estimate the frequency of gene targeting.

FIG. 5B shows examples of indels seen in moesina alleles.

FIG. 5C shows examples of indels seen in ppp1ca alleles.

FIG. 5D shows examples if indels seen in cdh5 alleles.

FIG. 6A shows the predicted exon and intron structure of the D. rerio gene for crhr2, LOC100335005. The vertical bold line represents a previously predicted exon from GENBANK® accession XM_{—}681362.3. The bar below the bold line represents the PCR amplicon used to assess mloxP integration.

FIG. 6B is a diagram showing TALE nuclease target sites (top), with the bar delineating the TALE nuclease binding sites with the spacer. In the middle of the diagram, the box represents the sequence of the crhr2 mLoxP injected oligonucleotide that serves as a template for HR. Sequences of the spacer region, the left homologous sequence, and the right homologous sequence are indicated. The modified loxP^{JTZ17} (mloxP) sequence is underlined. The lower two sequences indicate the sequences of wild-type crhr2 locus (WT) and the theoretical result of a precise integration of the mloxP oligonucleotide (HR). A gap of two nucleotides was placed in the WT sequence to allow vertical alignment of homology domains. Sequences that could be derived from the mLoxP oligonucleotide are capitalized.

FIG. 6C is a series of pictures of agarose gels showing PCR products derived from individual embryos. The number of embryos that incorporated the mLoxP sequence at the crhr2 locus, as indicated by a band at ˜225 bp, is listed for non-injected (WT), TALE nuclease mRNA only injected, and two ratios (2:2 and 2:1) of TALE nuclease mRNA and mLoxP oligonucleotides.

FIG. 6D (top) shows the sequence confirmation of three mloxP germline fish. One fish demonstrated precise germline HDR while two showed indels. In #N524, the reverse complement of the mloxP was observed (shaded in grey). FIG. 6D (bottom) shows individual sequences from the somatic tissue of distinct embryos. Sequence insertions are listed below with an arrow indicating the location of the insert.

FIG. 6E shows the individual sequences from four embryos. mloxP sequence integrations are underlined. The S# (S1, S2, etc.) indicates the place at which the indicated S# sequences were inserted.

FIG. 7 shows the amino acid sequence of the +63 second-generation scaffold (SEQ ID NO:104).

FIG. 8 shows the amino acid sequence of a TALE nuclease scaffold (SEQ ID NO:105).

FIG. 9 is a sequence alignment of SEQ ID NO:104 (bottom) and SEQ ID NO:105 (top).

FIG. 10 shows the repeat variable diresidues (RVDs) used in the final GoldyTALEN and Miller +63 constructs. HD, NG, NI, NN=RVD with corresponding di-residue at position 12-13.

FIG. 11 is a picture of a gel showing germline screening of the crhr2 locus. 53 adult fish were prescreened via fin biopsy. Of those prescreened, 20 demonstrated mloxP maintenance. 16 F0s were outcrossed with two exhibiting germline transmission. 42 unscreened F0s were outcrossed, and four exhibited germline transmission.

FIG. 12 is series of pictures of gels showing biallelic gene targeting in F0 GoldyTALEN-injected embryos. GoldyTALENs against the cdh5 gene were injected at the 1-cell stage, and embryos were allowed to develop to the 256-cell stage (FIG. 12A), 24 hours post-fertilization (hpf) (FIG. 12B), or 50 hpf (FIG. 12C).

FIG. 13 is a series of graphs and gels showing biallelic gene targeting in F0 GoldyTALEN-injected embryos with low doses of TALE nuclease mRNA. FIG. 13A is a series of graphs plotting phenotype after injection of GoldyTALENs at different mRNA doses. Twenty embryos in each group were scored at 2 dpf for a consistent phenotype. Fish were injected with moesina (top), ppp1cab (middle), and cdh5 (bottom) GoldyTALENs. FIG. 13B is pictures of gels showing the effects of low doses of GoldyTALENs against moesina (top), ppp1cab (middle), and cdh5 (bottom) at 2 dpf.

FIG. 14 is a pair of immunohistochemistry images showing cdh5 GoldyTALEN-injected embryos. Bottom, cdh5 GoldyTALEN-injected embryos had undetectable amounts of Cdh5 protein compared to uninjected embryos (top). n=nucleus.

FIG. 15 is a series of results showing somatic targeted genome editing using GoldyTALENs at the ponzr1 locus. FIG. 15A is a picture of a representative gel showing integration of an artificial EcoRV sequence into the ponzr1 locus. Cut bands demonstrate EcoRV sequence insertion. FIG. 15B is a sequence analysis showing somatic introduction of the engineered EcoRV into twelve independently modified ponzr1 chromosomes. The 7 base pair insert with the EcoRV site (GATATCC) is underlined. Insertion sequences at the 5′ end are indicated using an arrow, deletions are delineated with a colon (:). FIG. 15C is a picture of a gel demonstrating germline transmission of the EcoRV site.

FIG. 16 shows the sequences of ssDNA oligonucleotides designed to test for HDR. Different lengths of homology arms to the ponzr1 locus were tested. The first four sequences incorporated EcoRV into the genome, while the last three oligonucleotides were used to detect the engineered mloxP. The homology arms of the first (20/18) went halfway through the TALE nuclease binding site. 48/45 indicates homology arms that were lengthened to include the entire TALE nuclease binding site. Bold type indicates mutations that were introduced to one side of the longer homology arms to prevent potential binding of the TALE nucleases to the ssDNA oligonucleotide. Long homology arms with mutations were made for either the 5′ (55/18) or 3′ (23/48) end. The column on the right indicates that percent of somatic sequence integration, with the number of positive embryos versus total embryos tested in parenthesis.

### DETAILED DESCRIPTION

This document provides methods and materials for genome editing and functional genomic applications. As described herein, TALE nucleases and donor nucleic acid can be used to perform homologous recombination successfully in species such as zebrafish. For example, this document demonstrates the ability to introduce genetic changes precisely at a TALE nuclease cut site in vivo using single-stranded DNA oligonucleotides as a donor sequence. Such methods can be used to introduce changes (e.g., small changes) at a TALE nuclease cut site in zebrafish. In some cases, the methods and materials provided herein can be used to introduce loxP-related sequences at two different TALE nuclease cut sites, thereby allowing for conditional allele generation in zebrafish and other species (e.g., other model systems). In some cases, single-stranded DNA (ssDNA) oligonucleotides (oligos) can be used to add new sequences successfully and precisely at predefined locations in the genome of a species (e.g., a zebrafish). In some cases, such an introduced sequence can be a modified loxP (mloxP) sequence as described elsewhere (Thomson et al., Genesis, 36:162-167, 2003).

Zinc finger nucleases (ZFNs) and TALE nucleases can be effective at introducing locus-specific double-stranded breaks in the zebrafish (Doyon et al., Nature Biotechnol., 26:702-708, 2008; Meng et al., Nature Biotechnol., 26:695-701, 2008; Foley et al., PLoS One, 4:e4348, 2009; Huang et al., Nature Biotechnol., 29:699-700, 2011; and Sander et al., Nature Biotechnol., 29:697-698, 2011), generating an array of small genome insertions or deletions including loss of function alleles. However, the efficacy of previously described custom restriction enzymes can be relatively low and can yield many unperturbed loci.

As described herein, synthetic ssDNA oligonucleotides can be used with a TALE nuclease system for genome editing including the precise introduction of exogenous DNA sequence at a specific locus, such as the addition of loxP sequences for the generation of conditional alleles. Although deployed here in zebrafish, this approach has the potential to be effective for in vivo applications in a wide array of model organisms (e.g., insects, nematodes, frogs, mice, rats, and rabbits).

Transcription activator-like (TAL) effectors are polypeptides of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors also can be engineered and generated for the purpose of binding to particular nucleotide sequences, as described herein.

For TALE nuclease polypeptides, a TAL effector can be fused to a nuclease or a portion of a nuclease, typically a nonspecific cleavage domain from a type II restriction endonuclease such as FokI (Kim et al. Proc. Natl. Acad. Sci. USA, 93:1156-1160, 1996). Other useful endonucleases may include, for example, HhaI, HindIII, NotI, BbvCI, EcoRI, BgtI, and AlwI. The fact that some endonucleases (e.g., FokI) function as dimers can be capitalized upon to enhance the target specificity of the TAL effector. For example, in some cases, each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

Sequence-specific TALE nucleases can be designed to recognize preselected target nucleotide sequences present in a cell. In some cases, a target nucleotide sequence can be scanned for nuclease recognition sites, and a particular nuclease can be selected based on the target sequence. In some cases, a TALE nuclease can be engineered to target a particular cellular sequence. A nucleotide sequence encoding the desired TALE nuclease can be inserted into any suitable expression vector, and can be operably linked to one or more promoters or other expression control sequences.

Examples and further descriptions of TALE nucleases can be found, for example, in U.S. Patent Application Publication No. 2011/0145940, which is incorporated herein by reference in its entirety. In some cases, as described herein, a TALE nuclease can have truncations at the N- and/or C-terminal regions of the TAL portion of the polypeptide, such that it has a shortened scaffold as compared to a wild type TAL polypeptide. An exemplary TALE nuclease with a modified scaffold is the +63 TALE nuclease described herein. It is to be noted that the TAL portion also can include one or more additional variations (e.g., substitutions, deletions, or additions) in combination with such N- and C-terminal scaffold truncations. For example, a TALE nuclease can have N- and C-terminal truncations of the TAL portion in combination with one or more amino acid substitutions (e.g., within the scaffold and/or within the repeat region).

Vectors comprising nucleic acid encoding a TALE nuclease can be introduced into cells by a variety of methods (e.g., injection, direct uptake, projectile bombardment, liposomes, or electroporation). As described in the Examples below, for example, DNA encoding a TALE nuclease can be microinjected into a zebrafish embryo. TALE nucleases can be stably or transiently expressed into cells using expression vectors. Techniques for expression in eukaryotic cells are well known to those in the art.

A donor nucleic acid also can be introduced into a cell, either simultaneously with or separately from the TALE nuclease nucleic acid. A donor nucleotide sequence (e.g., a single-stranded DNA (ssDNA) sequence) can, for example, include a variant sequence having one or more modifications (i.e., substitutions, deletions, insertions, or combinations thereof) with respect to a preselected target nucleotide sequence found endogenously within the genome of a cell to be transformed (also referred to herein as a “modified target nucleotide sequence”). In some cases, the donor nucleic acid can have an overall length that is from about 20 to about 90 nucleotides (e.g., 20 to 40, 20 to 45, 20 to 50, 20 to 55, 20 to 60, 25 to 40, 25 to 45, 25 to 50, 25 to 55, 25 to 60, 30 to 40, 30 to 45, 30 to 50, 30 to 55, 30 to 60, 30 to 65, 35 to 40, 35 to 45, 35 to 50, 35 to 55, 35 to 60, 35 to 65, 40 to 45, 40 to 50, 40 to 55, 40 to 60, 40 to 65, 40 to 70, 45 to 50, 45 to 55, 45 to 60, 45 to 65, 45 to 70, 45 to 74, 50 to 55, 50 to 60, 50 to 65, 50 to 70, or 50 to 75 nucleotides). In some cases, the variant sequence within a donor nucleic acid can be flanked on both sides with sequences that are similar or identical to the endogenous target nucleotide sequence within the cell. For example, the flanking sequences can have a length between about 10 and about 45 nucleotides (e.g., 10 to 30, 10 to 35, 10 to 40, 10 to 45, 15 to 30, 15 to 35, 15 to 40, 15 to 45, 20 to 35, 20 to 40, 20 to 45, 25 to 40, or 25 to 45 nucleotides), such that the overall length of the donor sequence is from about 20 to about 90 nucleotides (e.g., 20 to 40, 20 to 45, 20 to 50, 20 to 55, 20 to 60, 25 to 40, 25 to 45, 25 to 50, 25 to 55, 25 to 60, 30 to 40, 30 to 45, 30 to 50, 30 to 55, 30 to 60, 30 to 65, 35 to 40, 35 to 45, 35 to 50, 35 to 55, 35 to 60, 35 to 65, 40 to 45, 40 to 50, 40 to 55, 40 to 60, 40 to 65, 40 to 70, 45 to 50, 45 to 55, 45 to 60, 45 to 65, 45 to 70, 45 to 74, 50 to 55, 50 to 60, 50 to 65, 50 to 70, or 50 to 75 nucleotides). In some cases, homologous recombination can occur between the donor nucleic acid and the endogenous target on both sides of the variant sequence, such that the resulting cell's genome contains the variant sequence within the context of endogenous sequences from, for example, the same gene. A donor nucleotide sequence can be generated to target any suitable sequence within a genome.

Methods for altering the genetic material of an organism can include introducing a TALE nuclease into a cell of the organism, either by introducing a TALE nuclease polypeptide or by introducing a nucleic acid encoding such a TALE nuclease polypeptide. In some cases, a method provided herein can include introducing both a TALE nuclease and a heterologous donor nucleic acid into a cell. The donor nucleotide sequence can include one or more modifications (i.e., substitutions, deletions, insertions, or combinations thereof) with respect to a corresponding, preselected target nucleotide sequence found in the cell. The donor nucleotide sequence can undergo homologous recombination with the endogenous target nucleotide sequence, such that the endogenous sequence or a portion thereof is replaced with the donor sequence or a portion thereof. The target nucleotide sequence typically includes or is adjacent to a recognition site for a sequence-specific TALE nuclease. In some cases, a target nucleotide sequence can include recognition sites for two or more distinct TALE nucleases (e.g., two opposed target sequences that are distinct, such that TALE nucleases having distinct DNA sequence binding specificity can be used). In such cases, the specificity of DNA cleavage can be increased as compared to cases in which only one target sequence (or multiple copies of the same target sequence) is used. In some cases, the donor nucleotide sequence and the nucleotide sequence encoding the TALE nuclease can be contained in the same nucleic acid construct. In some cases, the donor nucleotide sequence and the TALE nuclease coding sequence can be contained in separate constructs, or the TALE nuclease polypeptide can be produced and introduced directly into a cell.

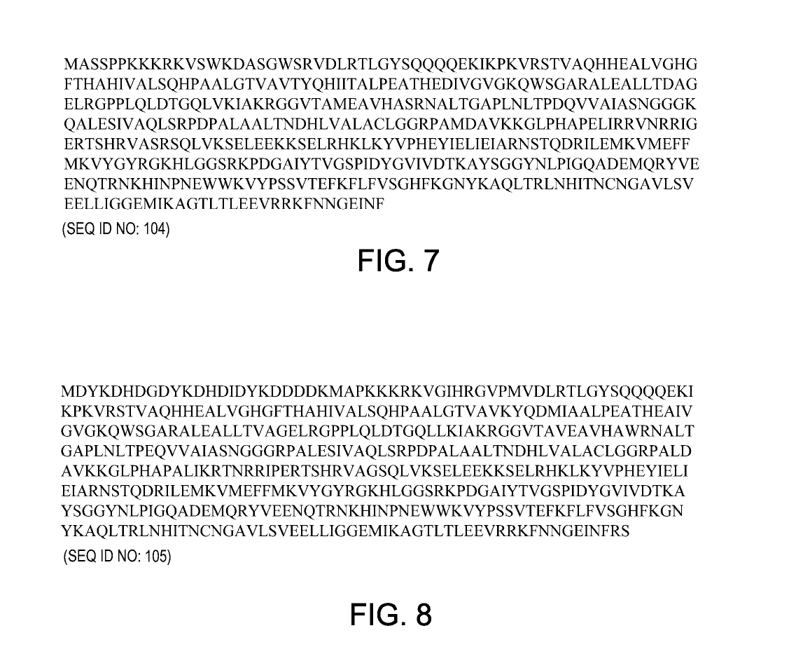

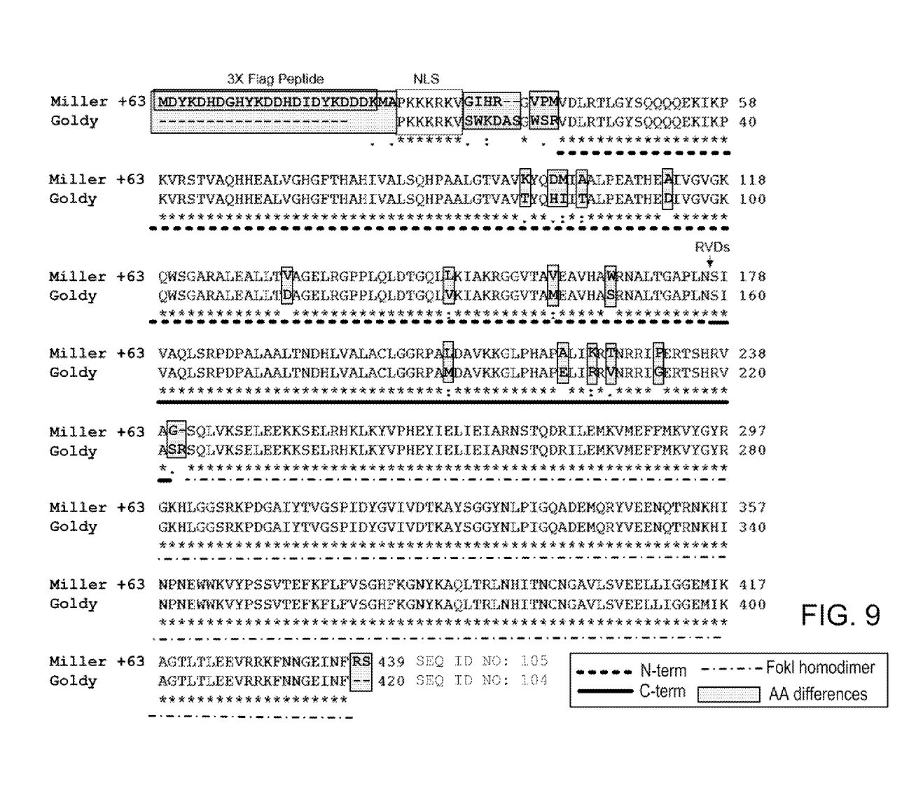

Any appropriate TALE nuclease can be used as described herein. In some cases, a TALE nuclease having a scaffold based on or including SEQ ID NO:104 or SEQ ID NO:105 can be used (see, e.g., FIGS. 7-9).

The invention will be further described in the following examples, which do not limit the scope of the invention described in the claims.

### EXAMPLES

### Example 1

### Materials and Methods

TALE Nuclease Design:

Software available at https://boglab.plp.iastate.edu/node/add/talen was initially used to find candidate binding sites. Three criteria were used for TALE nuclease design. First, repeat arrays that ranged from 15-25 bases in length were selected. Second, the spacer length was restricted to 14 or 18 bp, with 15-16 bases being the optimum length. Finally, if possible, one restriction enzyme site was present within that spacer.

To streamline the design process, a computer program was devised and implemented to aid in the design of TALE nucleases. This program works through multiple steps to find good TALE nuclease binding sites. First, the user supplies an NCBI Gene identifier and the program downloaded the sequence. The program then uses NCBI EUtilities to extract the exons with some flanking sequence. The flanking sequence is needed if the exons are short or the TALE nuclease recognition sequence is near the beginning or end of the exon. TAL binding sites are located using the required criteria of a thymine on either end of the binding sequence (T [ACG] [TCG] . . . T) as described by Moscou and Bogdanove (Science, 326:1501, 2009) and Cermak et al. (Nucl. Acids Res., 39:e82, 2011). The final thymine is required because of the B plasmid's design in the Golden Gate TALEN and TAL Effector kit (Addgene, 1000000016, USA). Once the TALE nuclease binding sites are found, the program locates any commercially available restriction enzymes that cut within the spacer region. When a restriction site is located, the program scans for 300 base pairs surrounding the TALE nuclease binding sites and reports only those enzymes that cut once or twice in the amplicon. The results, including the TALE nuclease binding sequences and the restriction enzymes that cut within the spacer, are reported in an easily usable format. The program source is freely available at zfishbook.org/tal_tool and, for convenience, also can be accessed via a web-based interface.

TALE Nuclease Binding Sites and Spacer Regions:

The following TALE nuclease recognition sites and spacer sequences were used:

ponzr1
left TALE nuclease
(SEQ ID NO: 124)
5′-GTGAGCACCCAGCGGACCTCCTCT-3′
right TALE nuclease
(SEQ ID NO: 125)
5′-ATCAGAACAACAGTCAGAGAT-3′
18 bp spacer
(SEQ ID NO: 126)
5′-GGAACCTGGACCACGGGC-3′
with a BstNI site (underlined)
crhr1
left TALE nuclease
(SEQ ID NO: 127)
5′-TGCAACACTGAGCTCTGTAAACCT-3′
right TALE nuclease
(SEQ ID NO: 128)
5′-CTGCTGCCGACTGGACCCTGAGGT-3′
15 bp spacer
(SEQ ID NO: 129)
5′-GTCCGCGTGTGGCGA-3′
with a BstUI site (underlined)
moesina
left TALE nuclease
(SEQ ID NO: 130)
5′-ACCCAGAAGACGTTT-3′
right TALE nuclease
(SEQ ID NO: 131)
5′-CTTTGAGTGGCCTCCT-3′
15 bp spacer
(SEQ ID NO: 132)
5′-CTGAGGAACTGATTC-3′
with an XmnI site (underlined)
ppp1cab
left TALE nuclease
(SEQ ID NO: 133)
5′-CTCCTCAACATACATACT-3′
right TALE nuclease
(SEQ ID NO: 134)
5′-GCCTCTGTCAACATAGT-3′
14 bp spacer
(SEQ ID NO: 135)
5′-CCTATTTCTGGGAG-3′
with a BsII site (underlined)
cdh5
left TALE nuclease
(SEQ ID NO: 136)
5′-CTCCTCAACATACATACT-3′
right TALE nuclease
(SEQ ID NO: 137)
5′-ACAAATGATTCATCTT-3′
16 bp spacer
(SEQ ID NO: 138)
5′-GGAGAGTTAGTTGACA-3′
with a HincII site (underlined)
crhr2
left TALE nuclease
(SEQ ID NO: 139)
5′-GTCAAATCTGCAGCTCCACGCTT-3′
right TALE nuclease
(SEQ ID NO: 140)
5′-CCTCTGCCTCTGACTCTGT-3′
15 bp spacer
(SEQ ID NO: 141)
5′-CACGCCTCAGCAAAC-3′

TALE Nuclease Constructs:

TALE nuclease assembly of the RVDs was performed using the Golden Gate approach as previously described by Cermak et al. (supra). Once assembled, rather than using the kit's destination vector, the RVDs were added to two different vectors—pT3 Ts-TAL+231 and pT3 Ts-TAL+63—which were used for in vitro transcription of TALE nuclease mRNA based on pT3TS vector previously described (Hyatt and Ekker, Meth. Cell Biol., 59:117-126, 1999). The TALE nuclease expression constructs were linearized with SacI, and mRNA was made (T3 mMessage Machine kit, Ambion) and purified (RNeasy MinElute Cleanup kit, Qiagen) for injection.

TALE Nuclease Germline Screening:

One-cell embryos were microinjected with 50-400 pg of TALE nuclease mRNA. Genomic DNA was collected at 4 dpf from 24-32 individual larvae as described in Meeker et al. (BioTechniques, 43:610, 612, and 614, 2007). Genomic DNA isolated from 10 larval zebrafish was extracted using DNAeasy Blood and Tissue kit (Qiagen). Genotyping was performed using PCR followed by restriction enzyme digest. The primers were as follows:

ponzr1
(SEQ ID NO: 142)
5′-GTTCACACAAAATGTCTCTCAAGTCTCTAAATC-3′
(SEQ ID NO: 143)
5′-AGTGGCCAGTGAGTGTATGTTACCT-3′
crhr1
(SEQ ID NO: 144)
5′-CGTGAAAGAGACAGCGAAGGGATTG-3′
(SEQ ID NO: 145)
5′-AGAAACTACCATTGTCACACTGAGCGAAG-3′
moesina
(SEQ ID NO: 146)
5′-GTTACGGCTCAAGACGTC-3′
(SEQ ID NO: 147)
5′-CAGGATGCCCTCTTTAAC-3′
ppp1cab
(SEQ ID NO: 148)
5′-GATGTTCATGGTCAGTAC-3′
(SEQ ID NO: 149)
5′-TGATTGAGGCACATTCATGG-3′
cdh5
(SEQ ID NO: 150)
5′-TTGTTGTCCTTGCAAAGCTG-3′
(SEQ ID NO: 151)
5′-TCTAGAGGATTCGCTGAT-3′
crhr2
(SEQ ID NO: 152)
5′-CCCTGATTGTGGAACTTTTCAGAACGTA-3′
(SEQ ID NO: 153)
5′-TGGTTTGGAATTAGTGCAGCATGAGTA-3′

The undigested bands were cloned into the TOPO® TA Cloning Kit (Invitrogen) and sequenced to confirm mutation.

Genome Editing:

For the ponzr1 loci, single-stranded DNA (ssDNA) oligos were designed to target the spacer sequence between the TALE nuclease cut sites. The oligo extended to half the length of the TALE nuclease recognition site. An EcoRV site (5′-GATSTCC-3′) or a mutated LoxP (mLoxP) site (5′-TAACTTCGTATAGCATACATTA TAGCAATTTAT-3′; SEQ ID NO:154) was introduced near the center of the oligo, resulting in a 20-base homology arm on the left side and an 18-base homology arm on the right side.

One-cell embryos were microinjected with 50-75 pg of ponzr1 or chrh2 TALE nuclease mRNA and 50-75 pg of one ssDNA donor. Genomic DNA was isolated as described above. If the embryos were injected with the EcoRV oligo, PCR was performed using the same primers as listed above and the product was digested using EcoRV. The positive larval DNA was cloned and colony PCR was used to find EcoRV-positive plasmids. Those plasmids were sent for sequencing to confirm EcoRV integration. If the embryos were injected with the mLoxP oligo, the genomic DNA was amplified using the same forward primer as listed above and a mLoxP reverse primer, 5′-ATAAATTGCTAT AATGTATGCTATACGAAGT-3′ (SEQ ID NO:155), or the same reverse primer as listed above and a mLoxP forward primer, 5′-ACTTCGTATAGCATA CATTATAGCAATTTAT-3′ (SEQ ID NO:156). The positive larval DNA was then amplified using the original pair of primers listed above and that product was cloned (TOPO® TA Cloning® Kit, Invitrogen) and colony PCR was used to find mLoxP-positive plasmids. The positive plasmids were sequenced for confirmation of mLoxP integration.

Zebrafish Work:

The zebrafish work was conducted under full animal care and use guidelines with prior approval by the local institutional animal care committee's approval. Danio rerio transgenic lines were described previously: Tg(fli1:EGFP) (Traver et al., Nat. Immunol., 4:1238-1246, 2003) and Tg(gata1:dsRed) (Lawson and Weinstein, Devel. Biol., 248:307-318, 2002).

Data Analysis and Statistics:

Quantification of TALE nuclease mutated DNA was performed using image J. For each gel, the background was subtracted and each lane was isolated to generate individual intensity plot profiles. A straight line was drawn across the bottom of each plot to eliminate inconsistencies caused by a skewed baseline. Each peak was then quantified. The intensity measurement for each band was added together to get total intensity. To calculate percent NHEJ, the intensity of the top band was divided by the total intensity. A student's T-test was used to test significance.

Cell-Free TALE Nuclease Restriction Endonuclease Assay:

5 ug of the ponzr1 PCR product was digested in each assay. Plasmids pTal 278, pTAL 279, pDelTal 278 and pDelTal 279 were linearized with SacI and used to transcribe messenger RNA using the mMessage RNA kit (Ambion). In vitro translation of 2 ug of each messenger RNA was accomplished using an In Vitro Transcription and Translation kit (Promega). ponzr1 PCR product was included in the assay mix during in vitro translation of different TALE nuclease combinations, allowing the translation and in vitro nuclease digestion to occur simultaneously. Translation was conducted for 2 hours at 30° C. To further facilitate TALE nuclease in vitro nuclease activity, the assay mix was diluted five-fold in in vitro digestion buffer (20 mM Tris-HCl pH 7.5, 5 mM MgCl₂, 50 mM KCl, 5% glycerol, and 0.5 mg/ml BSA). The assay mix was additionally incubated at 30° C. for 4 hours. DNA from the mix was purified using the Qiagen PCR Purification kit, concentrated via ethanol precipitation, and run on a 2% agarose gel. The negative control did not include the translated TALE nucleases.

### Example 2

### Results

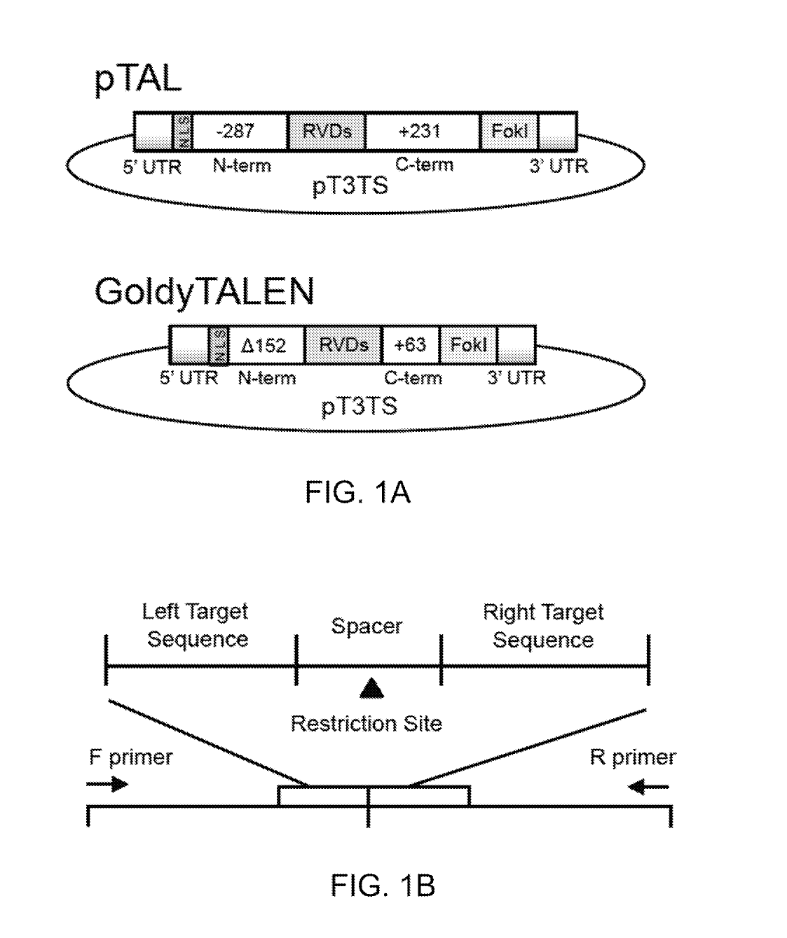

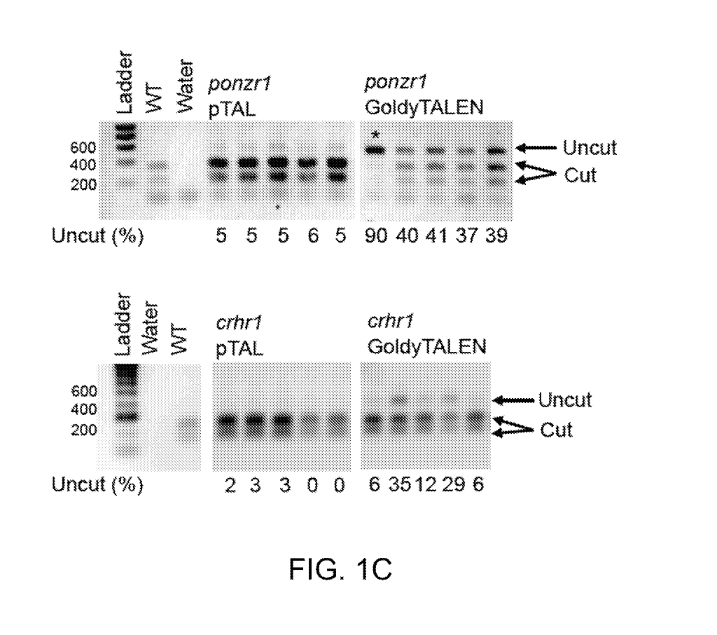

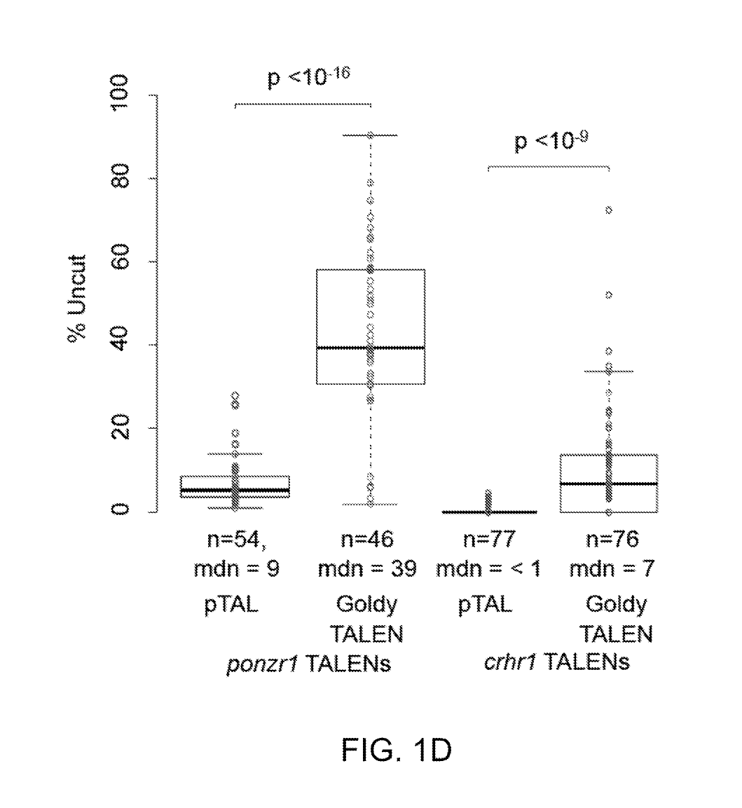

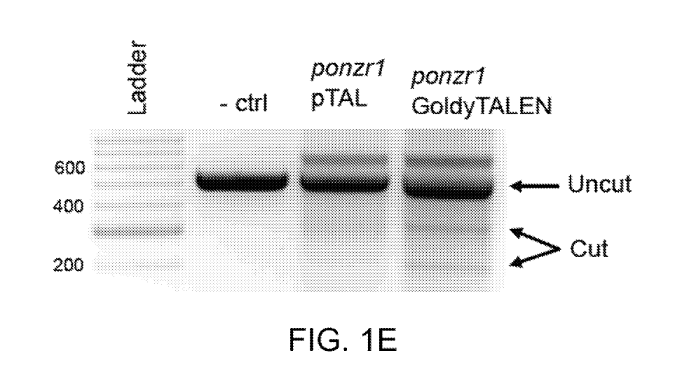

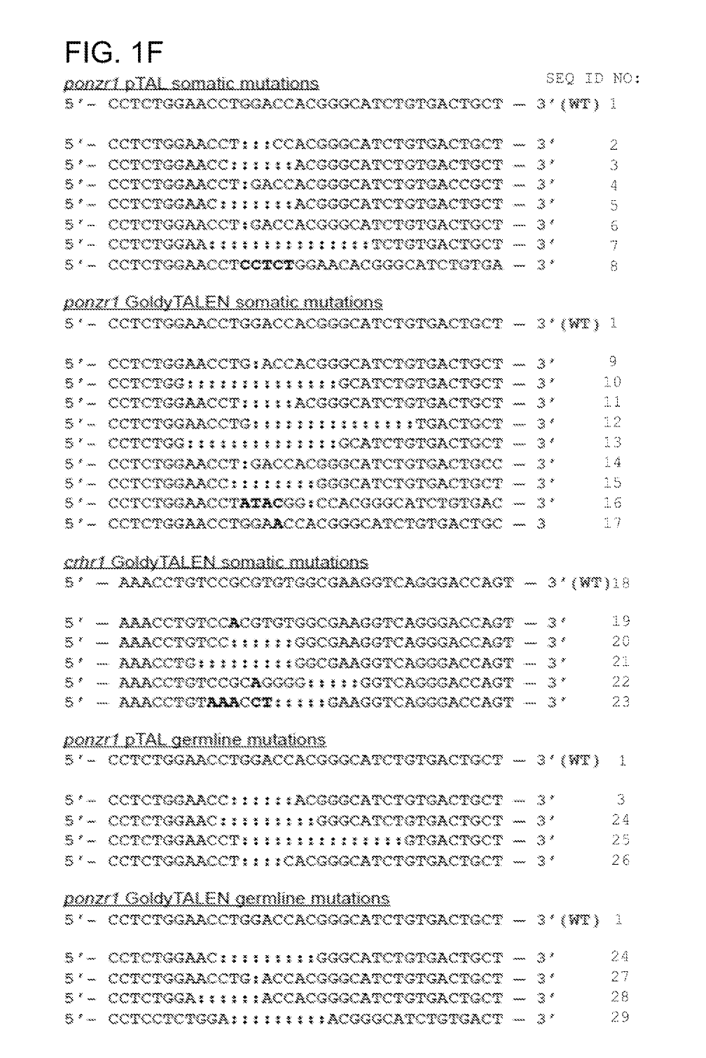

The efficacy of previously described custom restriction enzymes was relatively low and yielded many unperturbed loci (Doyon et al., supra; Meng et al., supra; Foley et al., supra; Huang et al., supra; and Sander et al., supra). For example, standard TALE nucleases using the +231 scaffold (FIG. 1A) targeting exon 2 of the zebrafish ponzr1 locus (Bedell et al., Development, 139:793-804, 2012) resulted in a readily measurable level of locus modification in somatic zebrafish tissue (median value of 5%; FIG. 1C). These genomic changes were typically small insertions/deletions (indels) indicative of error-prone non-homologous end joining (NHEJ or uncut) DNA repair (FIG. 1E). This TALE nuclease pair yielded four of 24 germline-transmitting founder animals carrying a mutation in ponzr1 (FIG. 1E). TALE nucleases against a second locus (crhr1) using a first generation scaffold yielded a very modest rate of sequence changes (<1%; FIG. 1C).

Different TALE nuclease scaffolds, with differential N- and C-terminal truncations, diverse linkers to the FokI nuclease, and distinct nuclear localization sequences, have been tested (Miller et al., Nature Biotechnol., 29:143-148 2011; Cermak et al., supra; and Mussolino et al., Nucl. Acids Res., 39(21):9283-9293, 2011). In the experiments described herein, a +63 scaffold was tested in an RNA expression vector backbone (FIG. 1A) for altered efficacy using the zebrafish in vivo assay system. Use of the same TAL sequences in the +63 TALE nuclease scaffold resulted in a 6-fold increase in sequence changes at the ponz1 locus (FIGS. 1C and 1D). A significant increase in efficacy also was detected using a cell-free assay system with in vitro translated TALE nuclease protein and purified DNA (FIG. 1E). The +63 TALE nucleases against crhr1 showed a substantial increase in genome modification, improving from <1% to 7% median cutting efficacy (FIGS. 1C and 1D). Sequence comparisons demonstrated small insertions and deletions at the cut site as a diagnostic for error-prone repair from NHEJ (FIG. 1F). The types of indels induced by TALs in the +63 scaffold were not detectably different than those from the standard +231 scaffold (FIG. 1F).

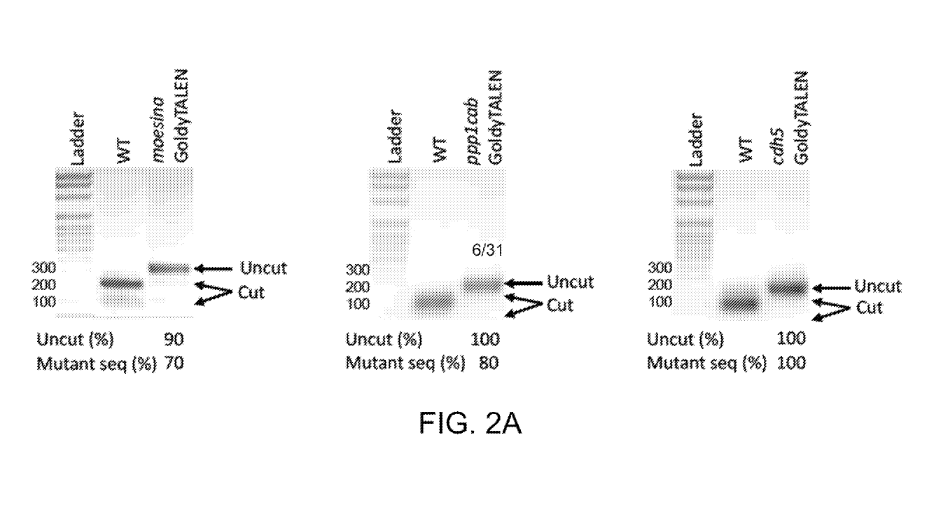

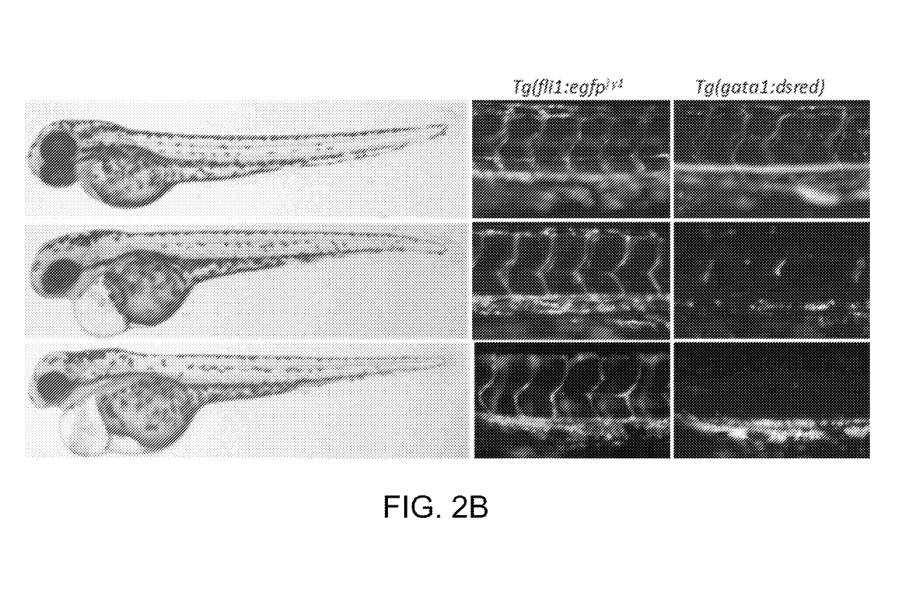

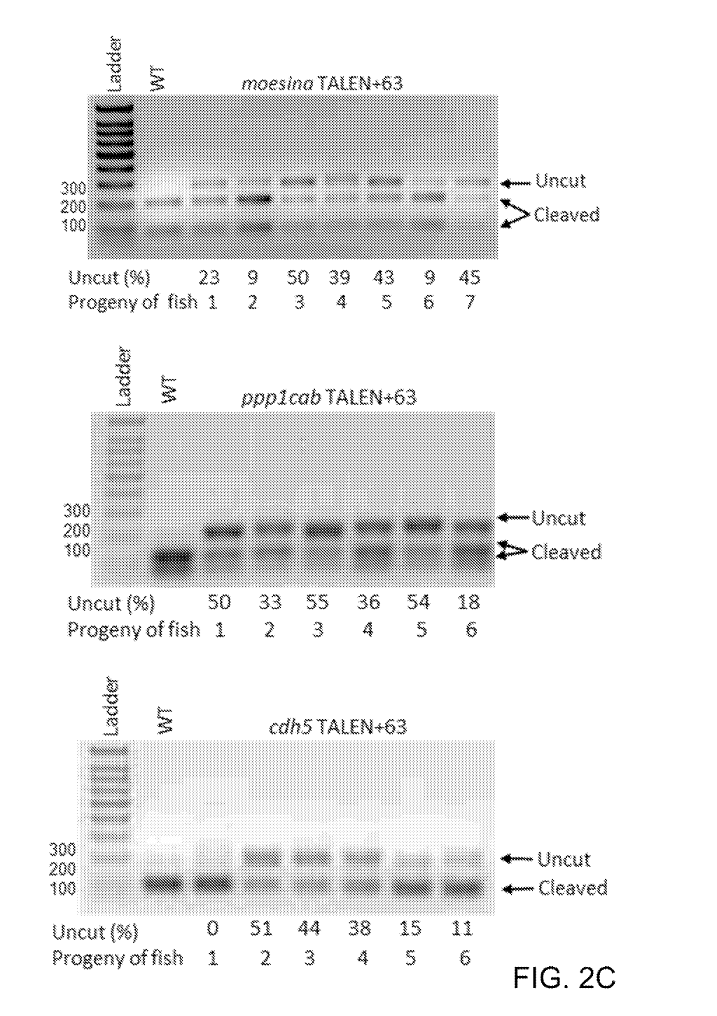

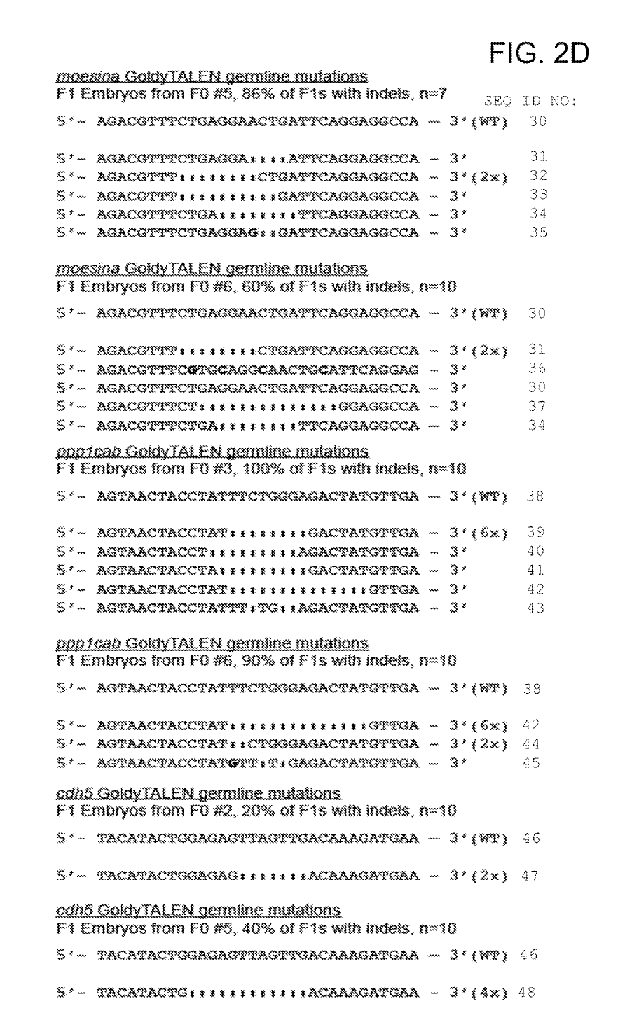

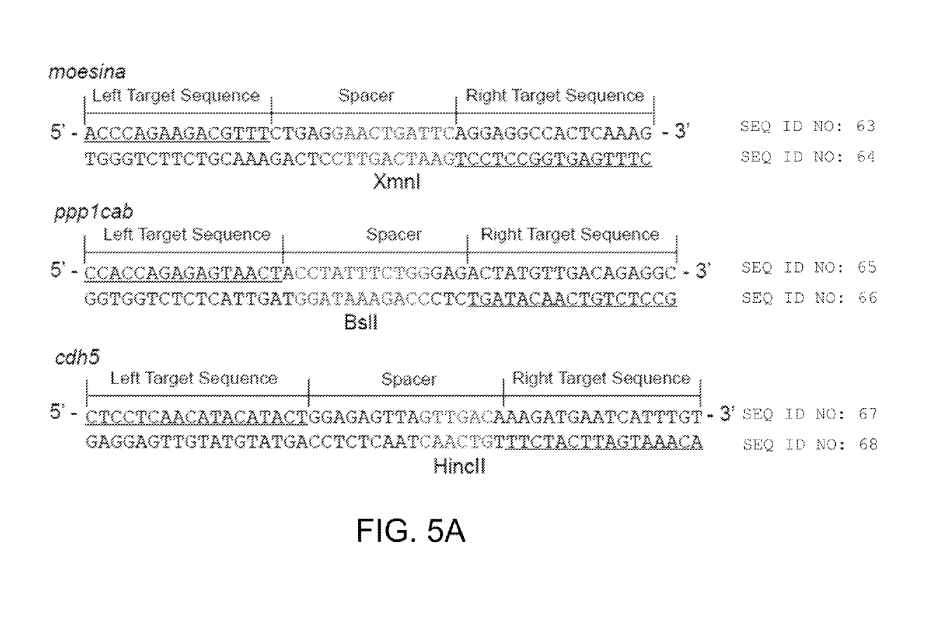

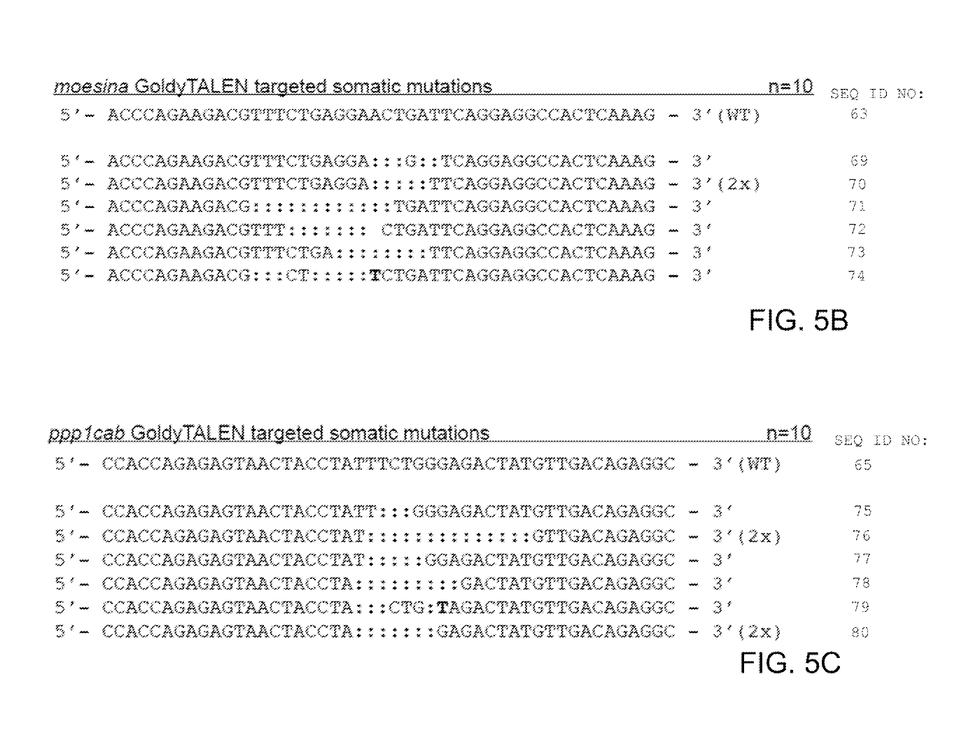

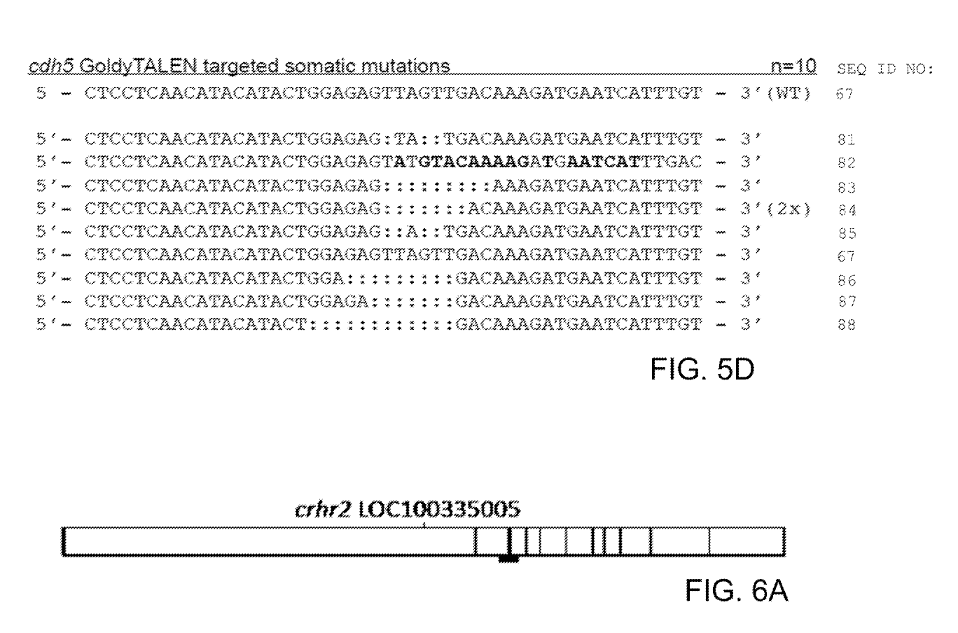

To further test the efficacy of the +63 scaffold, TALE nucleases were generated against three additional loci (moesina, ppp1cabb and cdh5; FIG. 5A). Efficient gene modification was observed at each locus (five out of five loci total; FIG. 1C and FIG. 2A). In three instances, the efficiency of mutagenesis ranged from 70 to 100% as demonstrated by loss of the restriction enzyme recognition sequence at the TALE nuclease cut sites (FIG. 2A) and direct DNA sequence analyses (FIGS. 5B-5D) of amplicons from pooled injected embryos. Together, these results indicate efficient gene targeting in somatic tissues that includes bi-allelic conversion in some animals. Somatic targeting efficacy using this second generation +63 scaffold compared favorably with previous reports of using TALE nucleases in zebrafish that showed amplicon restriction digest resistance between 2.4-12.4% (Huang et al., supra) and 11-33% by direct sequencing (Sander et al., supra) in F0 somatic tissue following injection. Thus, four of five +63 TALE nucleases resulted in a higher mutation frequency than any of the five previously reported loci using the first generation TALE nuclease systems.

Studies were then conducted to determine whether TALE nucleases could be used for targeted gene inhibition for phenotype studies using injected animals, such as targeted gene knockdown using morpholinos (MOs) (Nasevicius and Ekker, Nat. Genet., 25:216-220, 2000). Indeed, cdh5 TALE nuclease-injected larvae displayed specific vascular changes that phenocopied those generated by MOs (Wang et al., supra). Embryos injected with either cdh5 +63 TALE nucleases or MOs displayed similar vascular phenotypes: pronounced cardiac edema with blood pooling (FIG. 2B, left panels), grossly normal patterning of the trunk Tg(fli1-egfp)^{y1} vasculature (Lawson and Weinstein, supra) (FIG. 2B, center panels), but a lack of circulating Tg(gata1:dsred)^{sd2} red blood cells (Traver et al., supra) (FIG. 2B, right panels). Together, these results indicated that the +63 TALE nuclease platform can achieve efficient bi-allelic targeting resulting in specific loss-of-function phenotypes.

Germline transmission of +63 TALE nuclease-induced lesions also was very high (FIG. 2C). DNA analysis from 10 pooled F1 embryos obtained from outcrossed F0 founders displayed a 9 to 55% locus mutation frequency, with 50% being the theoretical limit. DNA analysis of 10 individual F1 embryos from outcrossed F0 fish carried a mutant allele in 20% to 100% of the F1 offspring (FIG. 2D). These data indicated that the efficient somatic targeting observed was effectively passed through the germline.

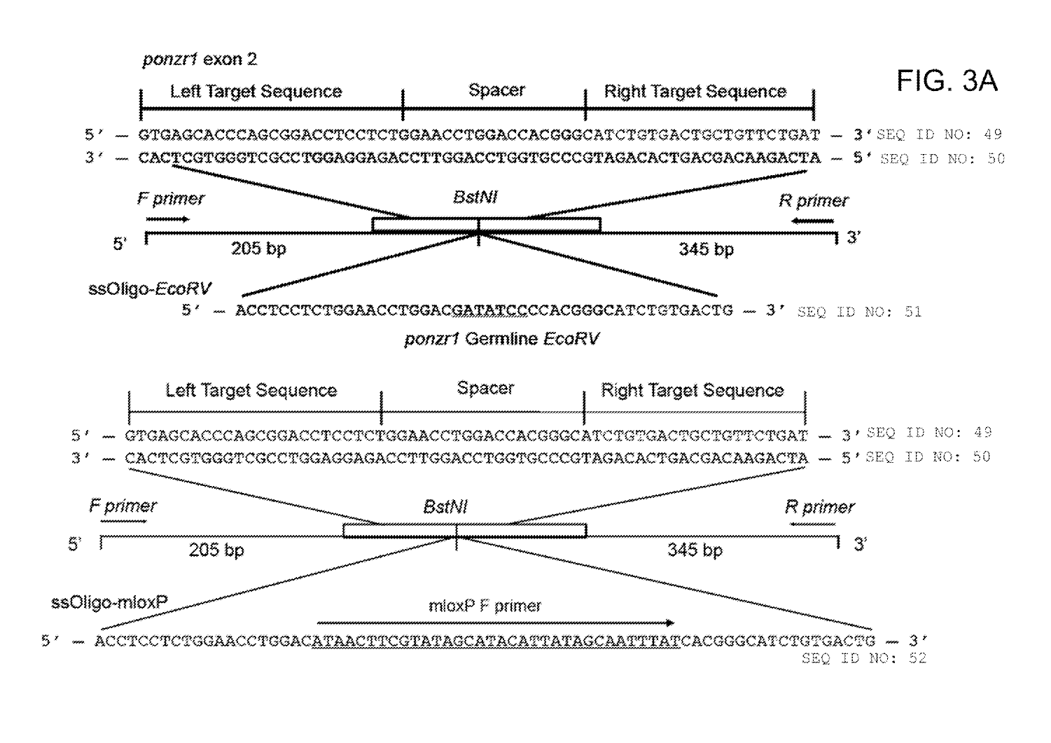

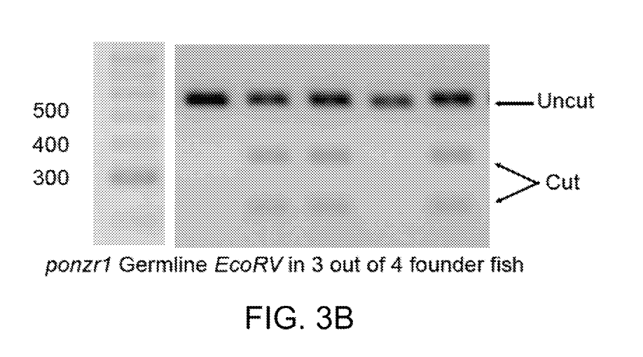

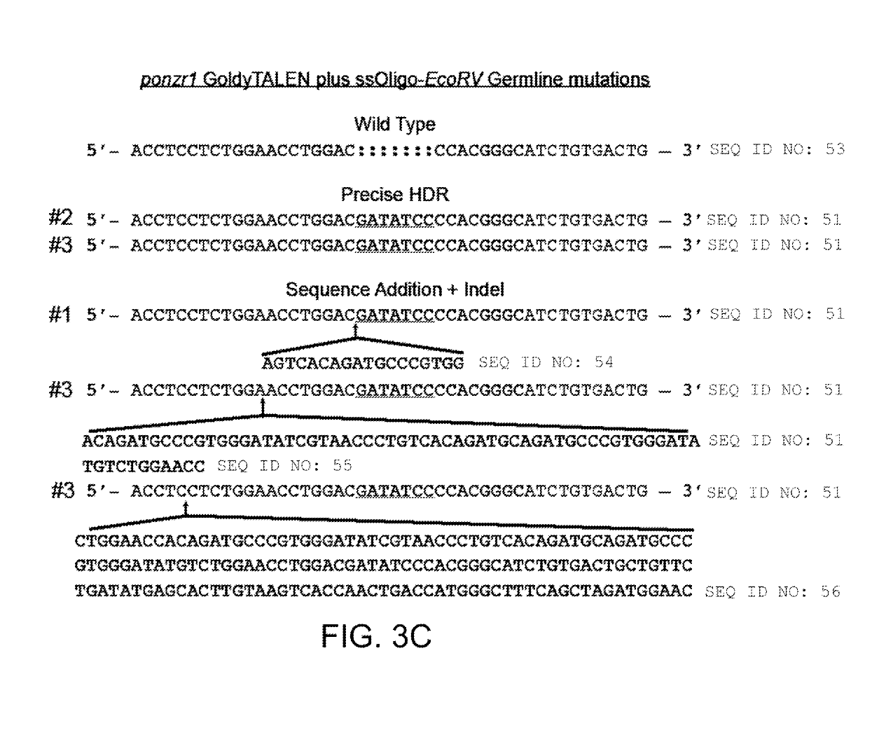

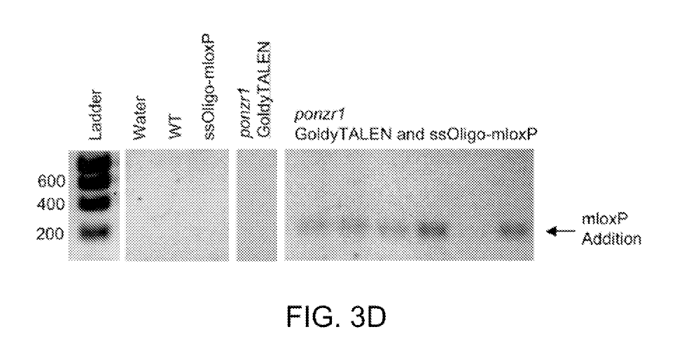

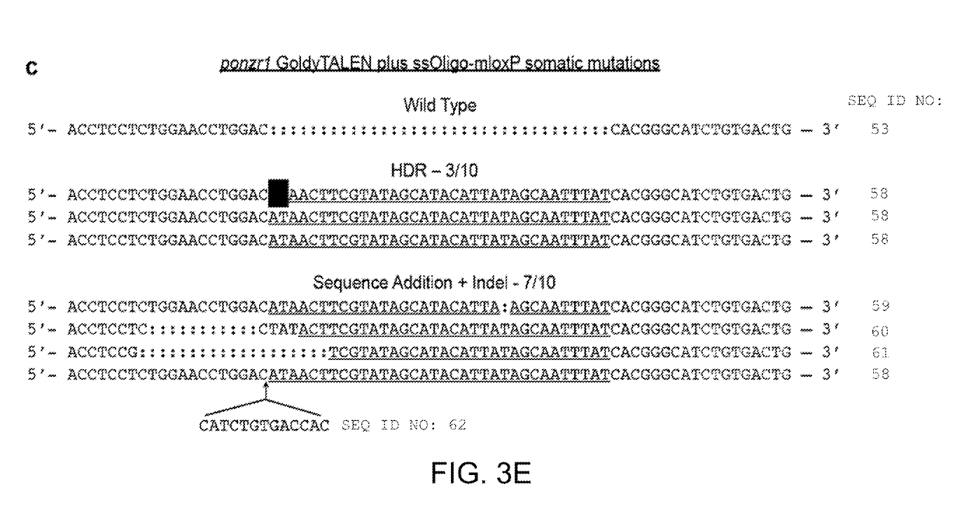

In vitro work has demonstrated that single-stranded (ss) DNA can be an effective donor for HR-mediated editing at a ZFN-induced double-stranded break (Chen et al., Nat. Meth., 8:753-755, 2011; and Porteus and Carroll, Nat. Biotechnol., 23:967-973, 2005). With the high-efficient genome modification success of +63 TALE nucleases, it was hypothesized that synthetic oligonucleotides designed to span the predicted TALE nuclease cut site could serve as an HR template in vivo (FIG. 3A). Using ponzr1 as a test locus, an EcoR V restriction site was effectively introduced by co-injection of ponzr1 TALE nucleases and a 45 base ssDNA oligo (FIG. 3B). In these experiments, 42 of 74 injected embryos displayed a detectable level of chromosomes containing the introduced EcoRV sequence, with an estimated 9% ratio of converted chromosomes in these animals (FIG. 3B). Sequence analysis indicated two precisely modified chromosome events from different zebrafish (FIG. 3C) demonstrating successful HR at the TALE nuclease cut site in ponzr1. Other events showed a precise addition at the 3′ end, while small indels were noted at the 5′ side of the modification site (FIG. 3C).

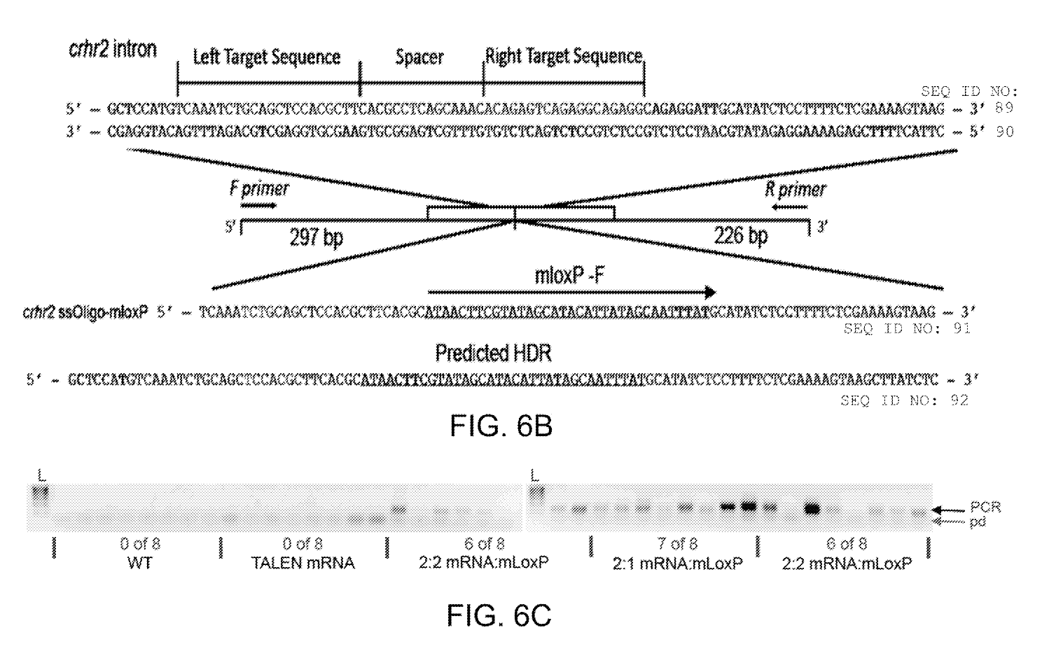

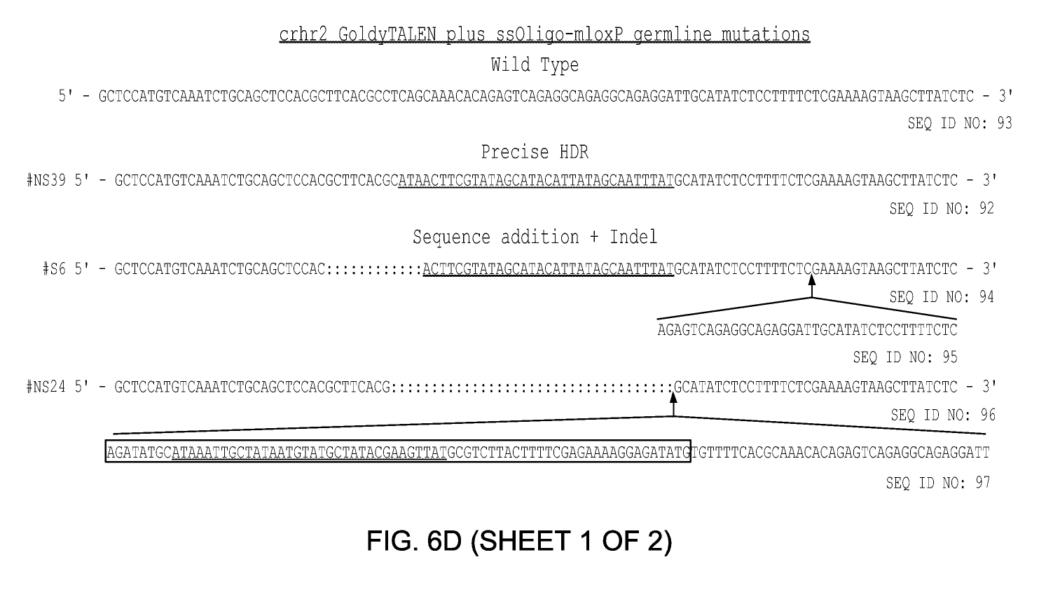

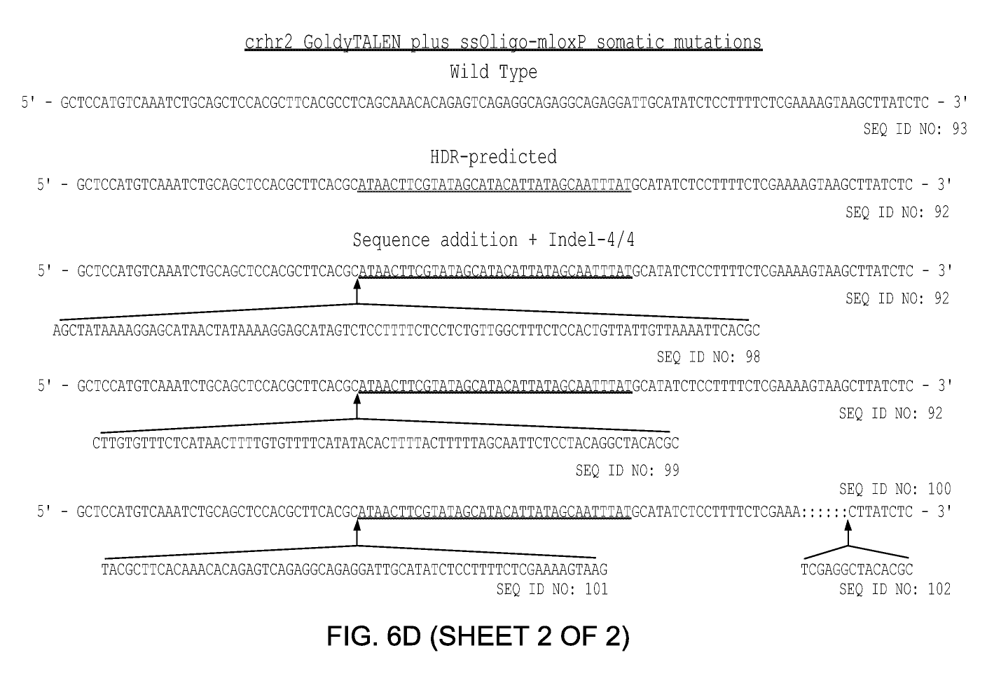

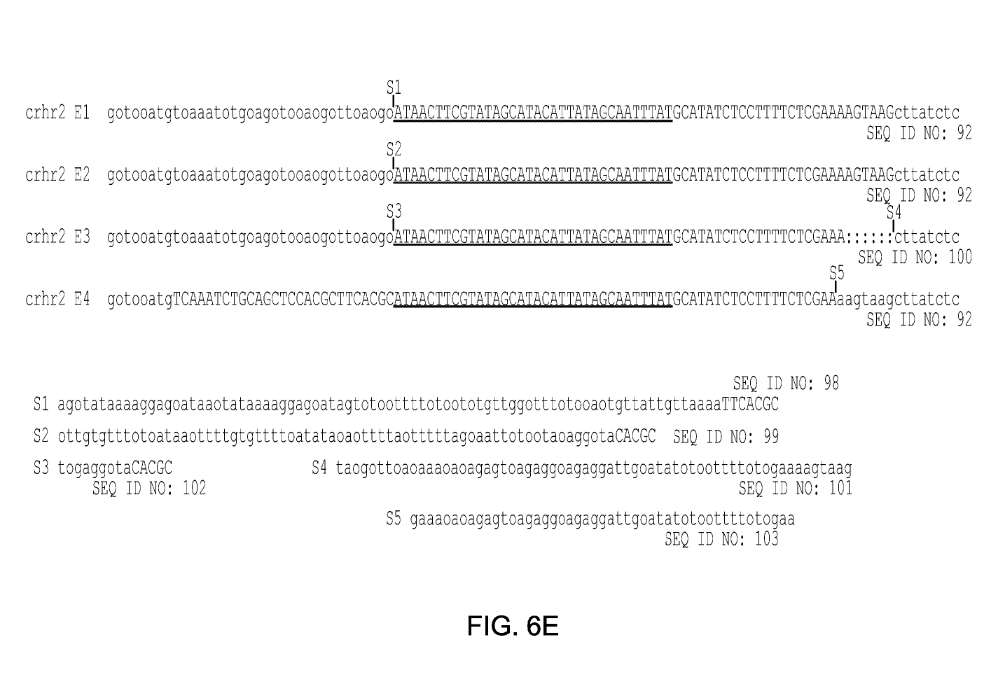

Studies were then conducted to investigate whether TALE nuclease/oligo co-injection could introduce larger sequences such as a loxP site, an essential step in making Cre-dependent conditional genetic alleles. TALE nucleases against the ponzr1 locus were used with long synthetic oligonucleotides to add a modified loxP^{JTZ17} (mloxP; Thomson et al., supra) site at this locus (FIG. 3A), a sequence designed to provide a single integration site for subsequent, Cre-mediated recombination into a genomic locus. PCR analysis demonstrated introduction of the mloxP sequence at the TALE nuclease cut site identifying 10 independent examples of the mloxP sequence at ponzr1 (FIG. 3D). Sequence characterization confirmed the precise integration of the mloxP site in three of 10 assayed chromosomes (FIG. 3E). The other seven events also resulted in the full integration of a mloxP site including a precise addition at the 3′ end while small indels were noted at the 5′ side of the TALE nuclease cut site. A similar method was used to introduce an mloxP sequence at a second location, the second intron of the crhr2 gene (FIG. 6A). Sequencing confirmed the genome addition at this locus as well (FIG. 6B). PCR products derived from individual embryos were analyzed, and the number of embryos that incorporated the mLoxP sequence at the crhr2 locus (indicated by a band at ˜225 bp) ranged from 0 of 8 for non-injected (WT) and TALE nuclease mRNA only injected, 6 of 8 for a 2:2 ratio of TALE nuclease mRNA:mLoxP oligos, and 7 of 8 for a 2:1 ratio of TALE nuclease mRNA:MLoxP oligos (FIG. 6C). The sequence was confirmed for three mloxP germline fish. One fish demonstrated precise germline HDR, while two showed indels (FIG. 6D, top). In #N524, the reverse complement of the mloxP was observed (shaded in grey). The bottom panel of FIG. 6D shows individual sequences from the somatic tissue of distinct embryos, with sequence insertions listed below each sequence with an arrow indicating the location of the insert. Individual sequences from four embryos are shown in FIG. 6E. The S# (S1, S2, etc.) indicates the place at which the indicated S# sequences were inserted

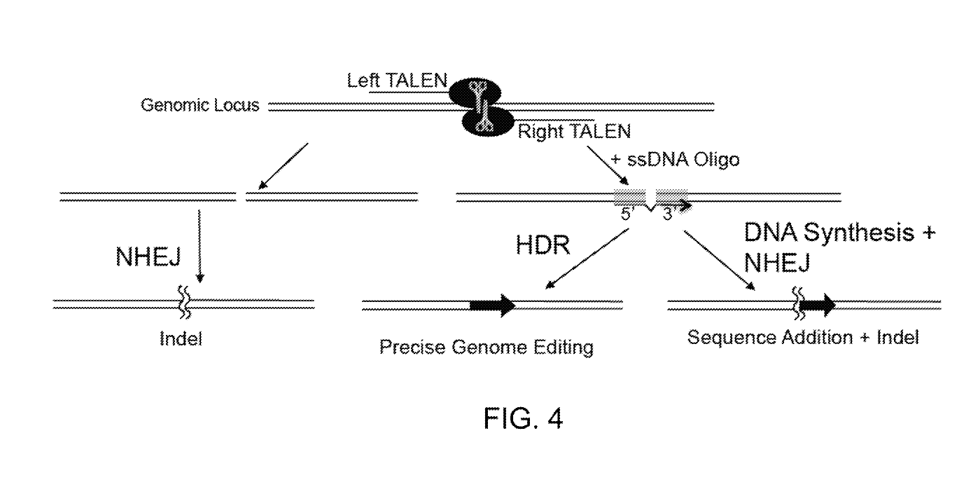

These results represent the first description of successful HR in zebrafish, and the first demonstration of HR using ssDNA as a donor template in vivo. This approach complements the established error-prone NHEJ toolkit for model organisms (FIG. 4). The use of ssDNA facilitates an array of genome changes, including the introduction of single nucleotide polymorphisms for vertebrate genetic applications. The asymmetry in precise editing that also was detected suggests an additional mechanism for genome editing that incorporates both HR and NHEJ (FIG. 4). For example, the donor ssDNA may serve as a primer for new strand synthesis at the TALE nuclease break. Extension from the 3′ end of the oligonucleotide would create long regions of homology for recombination. However, the 5′ end of the oligonucleotide places a limit on the extent of strand invasion from the genomic DNA and a limited opportunity for homologous recombination. This leads to resolution at the 5′ end by either HR or NHEJ. For applications where the new sequences are introduced to non-coding regions of the genome, such as the introduction of loxP sites in intronic sequences flanking an exon, either resolution event will likely be of high utility.

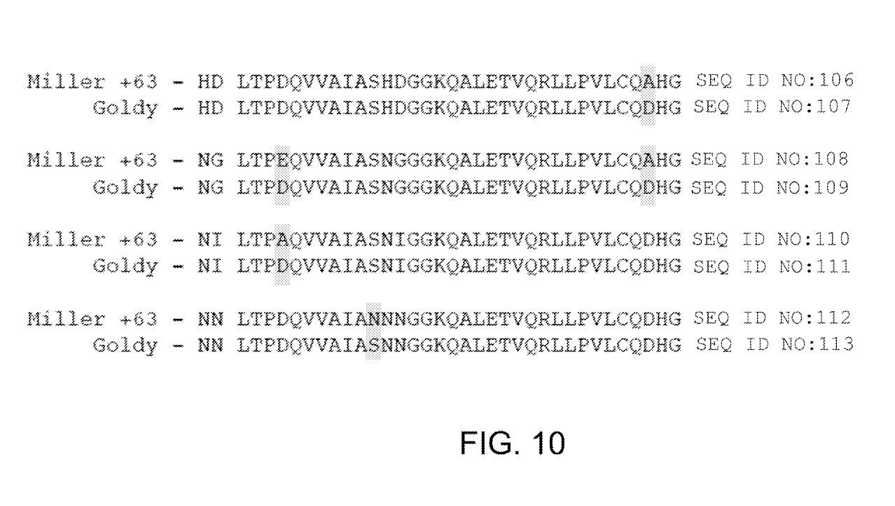

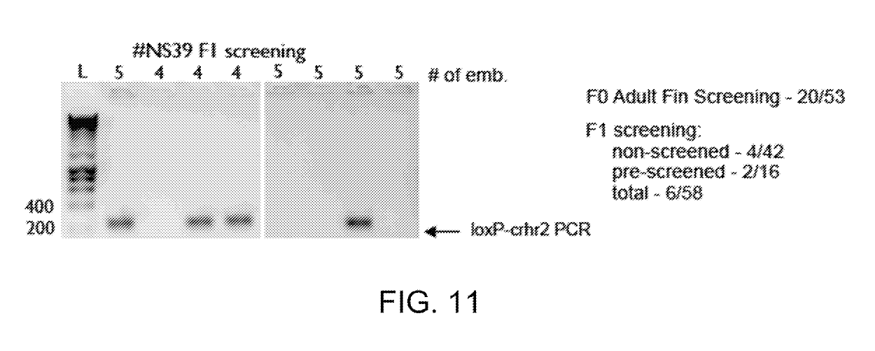

For the experiments described above, the RVDs used in the final GoldyTALEN and Miller +63 constructs are shown in FIG. 10. Amino acid differences arise from RVD variation in different Xanthomonas species. A representative gel from germline screening of the crhr2 locus is shown in FIG. 11. Of 53 adult fish that were prescreened via fin biopsy, 20 demonstrated mloxP maintenance. Sixteen F0s were outcrossed with two exhibiting germline transmission. Forty-two unscreened F0s were outcrossed, and four exhibited germline transmission.

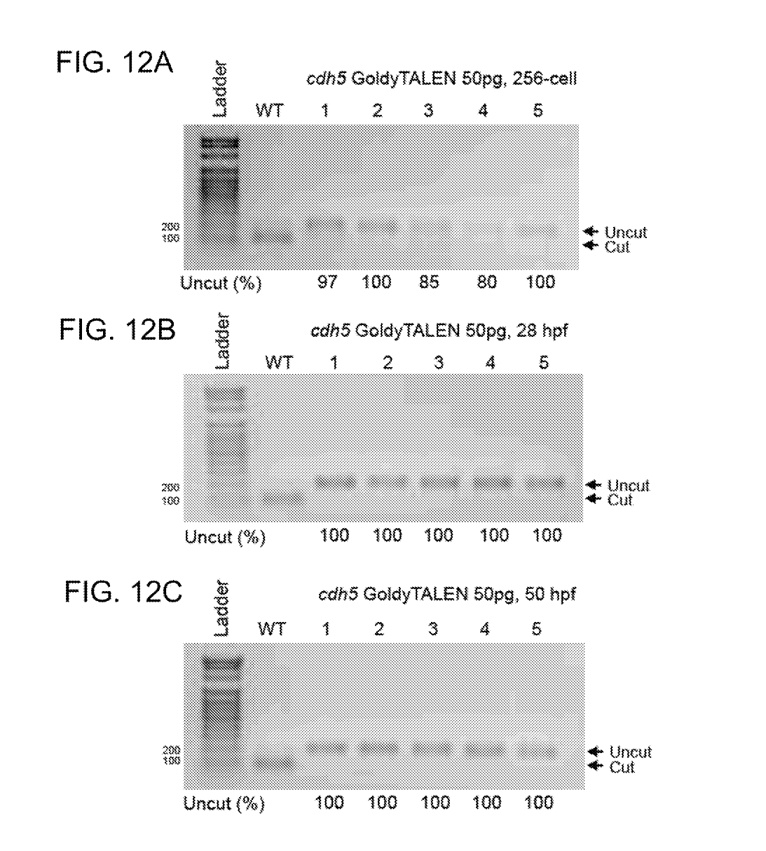

Biallelic gene targeting in F0 GoldyTALEN-injected embryos is depicted in FIG. 12. GoldyTALENs against the cdh5 gene were injected at the 1-cell stage, and embryos were allowed to develop to the 256-cell stage (FIG. 12A), 24 hours post fertilization (hpf) (FIG. 12B), or 50 hpf (FIG. 12C). DNA was isolated from groups of 10 embryos and assessed for loss of the HincII restriction endonuclease site in the amplicon. Gene targeting was incomplete at the 256-cell stage, but was complete by 24 hpf.

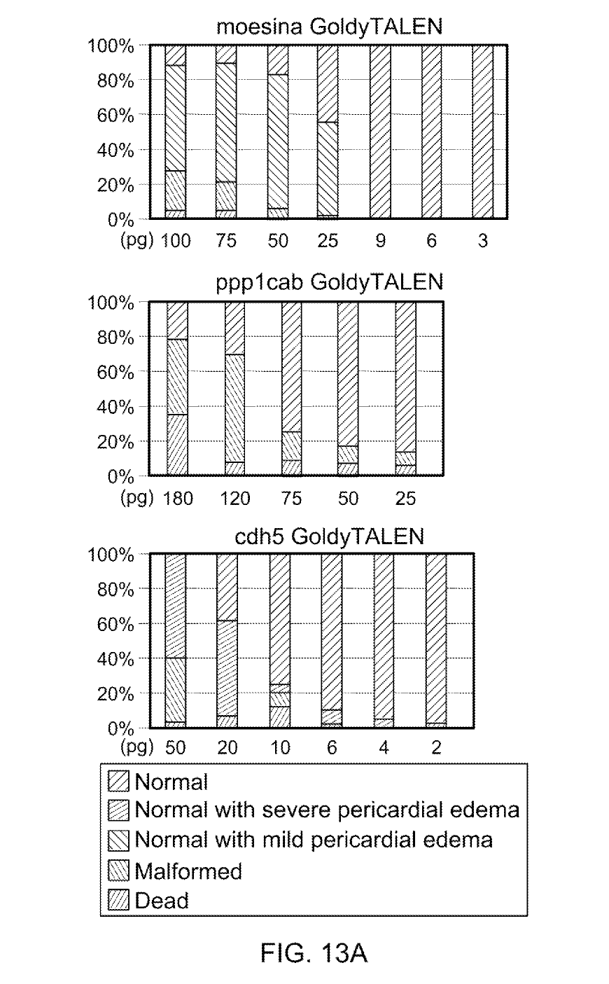

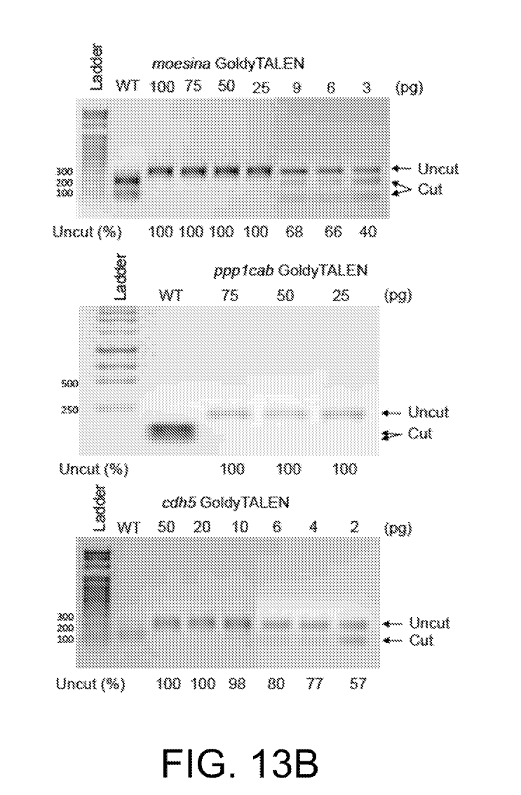

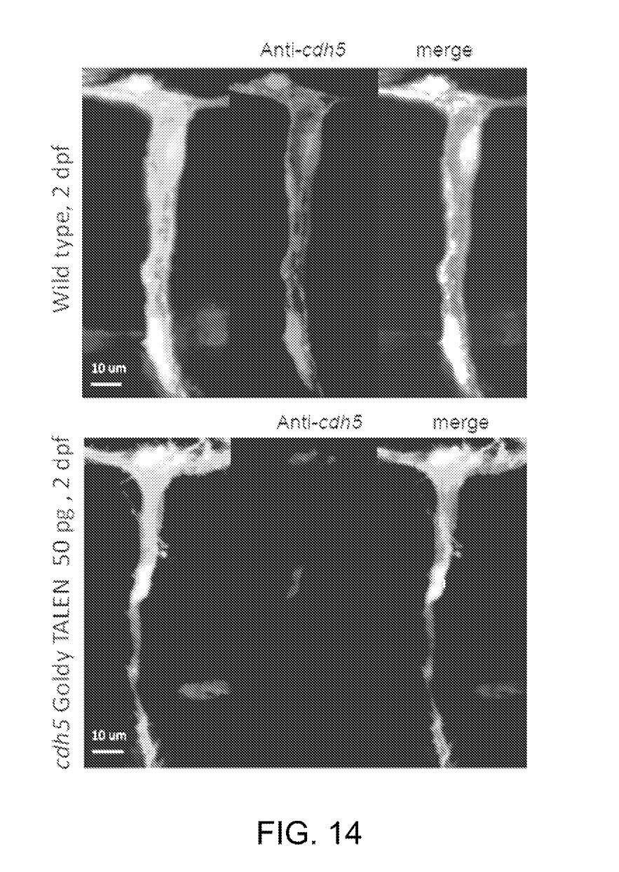

Biallelic gene targeting in F0 GoldyTALEN-injected embryos with low doses of TALE nuclease mRNA is shown in FIG. 13. GoldyTALENs were injected at different mRNA doses, and 20 embryos in each group were scored at 2 dpf for a consistent phenotype (FIG. 13A). In the moesina and cdh5 GoldyTALEN-injected fish (top and bottom graphs), pericardial edema that was associated with reduced circulation was observed. A consistent phenotype from injection of GoldyTALENs against ppp1cab was not noted (middle graph). Low doses of GoldyTALENs against for moesina (FIG. 13B, top), ppp1cab (middle), and cdh5 (bottom) resulted in biallelic gene targeting at 2 dpf. DNA was isolated from groups of ten embryos each for the concentrations indicated. Immunohistochemistry studies of cdh5 GoldyTALEN-injected embryos revealed a loss of Cdh5 protein in the intersegmental vessels of the zebrafish trunk, and showed that animals failed to undergo lumen formation (FIG. 14). Whole-mount immunohistochemistry revealed that cdh5 GoldyTALEN-injected embryos (bottom) had undetectable amounts of Cdh5 protein compared to uninjected embryos (top). These experiments were carried out in the Tg(fli1a:egfp)^{y1} line to allow visualization of the endothelial cells in the vasculature. Examination of the EGFP signals suggested defects in endothelial lumen formation, consistent with the lack of circulation observed in living embryos. A very low degree of mosaicism was observed with the absence of signal form the anti-Cdh5 antibodies in cdh5 GoldyTALEN-injected embryos. n=nucleus.

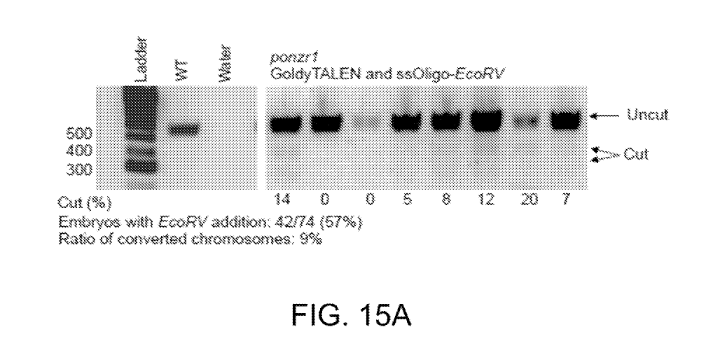

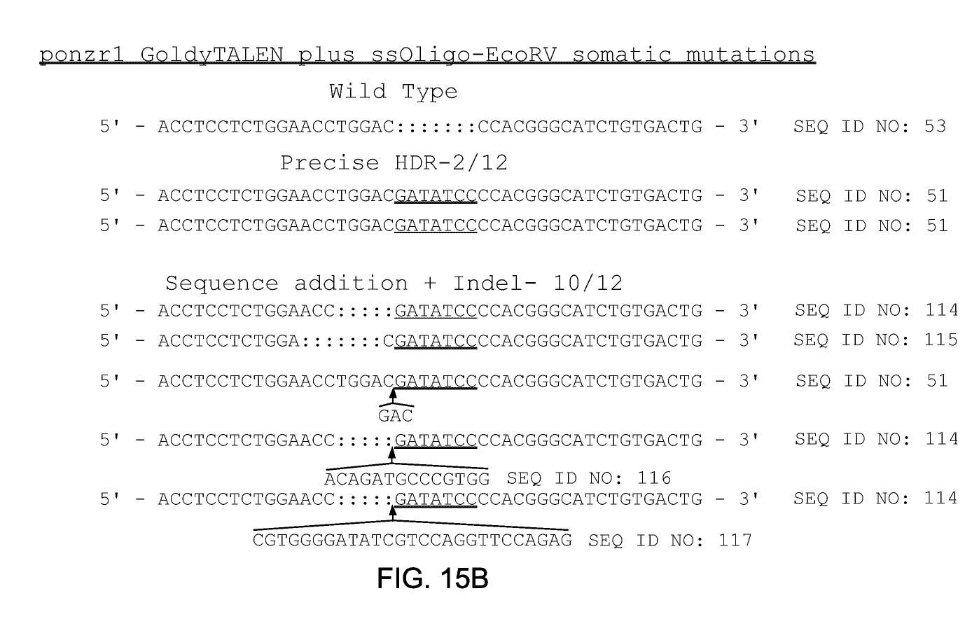

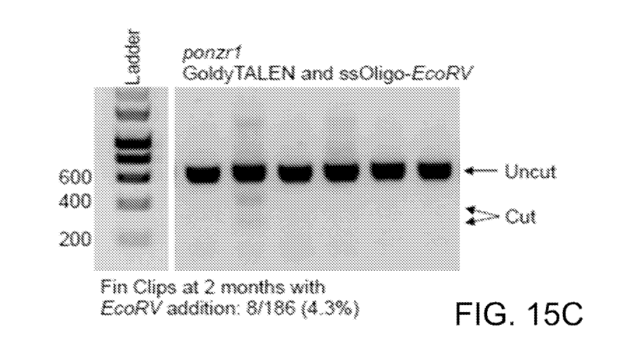

Targeted genome editing using GoldyTALENs at the ponzr1 locus is depicted in FIG. 15. An artificial EcoRV sequence integrated into the ponzr1 locus (FIG. 15A). Each lane shows a PCR-based DNA analysis of a single larva, and the cut bands demonstrated EcoRV sequence insertion. A sequence analysis confirming somatic introduction of the engineered EcoRV into twelve independently modified ponzr1 chromosomes is shown in FIG. 15B. Two of 12 sequenced clones demonstrated precise HDR, and 10 of the 12 embryos displayed an EcoRV insertion with indels on the 5′ end. Germline transmission of the EcoRV site was demonstrated (FIG. 15C). Fin biopsies were conducted on 2 month-old ssDNA plus ponzr1 GoldyTALEN-injected fish and analyzed for somatic EcoRV cutting. Eight of 186 demonstrated maintenance of the exogenous EcoRV sequence. Those fish were subsequently tested for germline transmission of the EcoRV site (FIG. 3B).

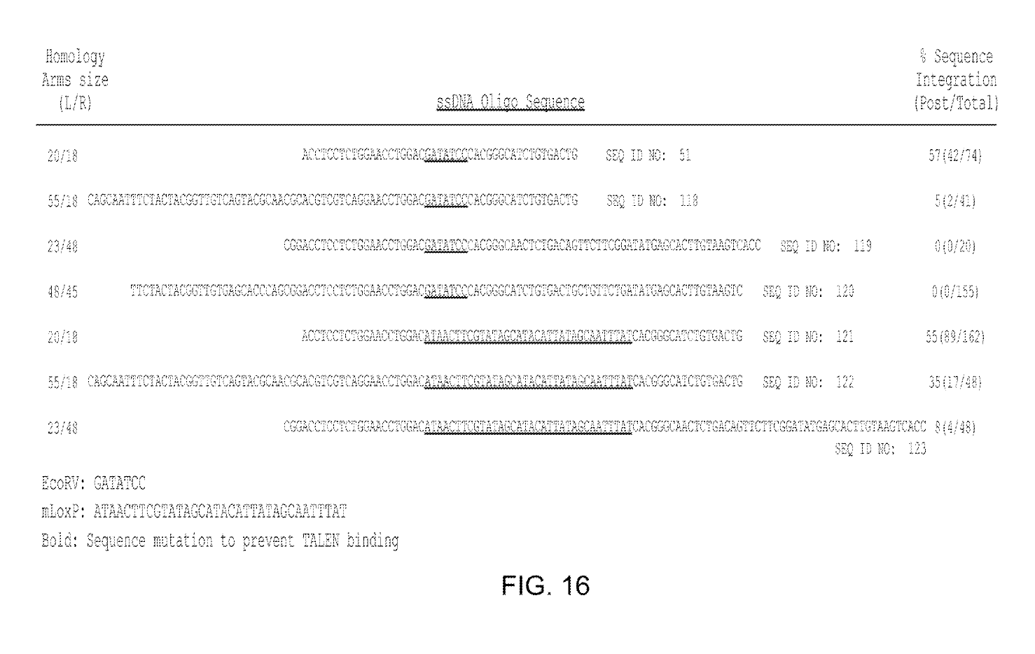

In further studies, single-stranded DNA oligonucleotides were designed to test for homology directed repair (HDR) in vivo. Different lengths of homology arms to the ponzr1 locus were tested. The first four sequences incorporated a novel seven-base sequence (GATATCC, underlined in FIG. 16) that includes an EcoRV restriction endonuclease recognition sequence. To screen, PCR was used to amplify the region, and samples were tested to look for EcoRV digestion. Sequence incorporation data is a measure of the fraction of injected embryos that displayed a measurable rate of EcoRV sequence incorporation; the threshold for detection was estimated to be between 1-5%. The last three oligonucleotides include incorporation of a modified Cre recombinase recognition sequence (mLoxP site, underlined in FIG. 16) with the same homology arms as the EcoRV oligos. These were screened using PCR with the reverse primer using the mLoxP site. This assay is much more sensitive at detecting DNA incorporation than the method used for the EcoRV HDR experiments. The homology arms of the first (20/18) incorporated half of the TALE nuclease binding site and showed significant HDR using both EcoRV and mLoxP oligos. When the homology arms were lengthened to include the entire TALE nuclease binding site (48/45), no HDR was seen and there was a decrease in TALE nuclease cutting. Oligos with mismatches (bold type in FIG. 16) designed to reduce any potential TALE nuclease binding were introduced to one side of the longer homology arms and the other side was kept halfway through the binding site. The long homology arms with mutations were made for either the 5′ (55/18) or 3′ (23/48) end. However, both of these oligos demonstrated significantly less sequence incorporation when compared to the 20/18 sequence. These results were surprising, because longer homology arms typically are thought to be required to achieve HDR. In contrast, the shortest constructs used in these experiments was the most effective.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for modifying the genetic material of an organism, comprising introducing into a cell of the organism:
(i) a first nucleic acid encoding a first transcription activator-like effector (TALE) nuclease monomer,
(ii) a second nucleic acid encoding a second TALE nuclease monomer, and
(iii) a donor nucleic acid,
wherein each of said first and second TALE nuclease monomers comprises a plurality of TALE repeat sequences and a FokI endonuclease domain, wherein said first TALE nuclease monomer comprises the ability to bind to a first half-site sequence of a target DNA within said cell and comprises the ability to cleave said target DNA when said second TALE nuclease monomer is bound to a second half-site sequence of said target DNA, wherein said target DNA comprises said first half-site sequence and said second half-site sequence separated by a spacer sequence, wherein said first and second half-sites have the same nucleotide sequence or different nucleotide sequences, and wherein said donor nucleic acid is from 35 to 55 nucleotides in length and comprises a sequence that is heterologous to said target DNA and that is flanked by sequences that are similar or identical to the endogenous target nucleotide sequence.

2. The method of claim 1, wherein the organism is a zebrafish embryo.

3. The method of claim 1, wherein the donor nucleic acid is a single stranded DNA.

4. The method of claim 1, wherein the donor nucleic acid is 40 to 50 nucleotides in length.

5. The method of claim 1, wherein said first TALE nuclease monomer, said second TALE nuclease monomer, or both said first and said second TALE nuclease monomers comprise a +63 scaffold backbone as set forth in SEQ ID NO:104.


SEQUENCE LISTING (156 sequences)

SEQ ID NO: 1 - DNA, 36 bp, Artificial Sequence
  synthetic oligonucleotide
cctctggaacctggaccacgggcatctgtgactgct

SEQ ID NO: 2 - DNA, 33 bp, Artificial Sequence
  synthetic oligonucleotide
cctctggaacctccacgggcatctgtgactgct

SEQ ID NO: 3 - DNA, 30 bp, Artificial Sequence
  synthetic oligonucleotide
cctctggaaccacgggcatctgtgactgct

SEQ ID NO: 4 - DNA, 35 bp, Artificial Sequence
  synthetic oligonucleotide
cctctggaacctgaccacgggcatctgtgaccgct

SEQ ID NO: 5 - DNA, 29 bp, Artificial Sequence
  synthetic oligonucleotide
cctctggaacacgggcatctgtgactgct

SEQ ID NO: 6 - DNA, 35 bp, Artificial Sequence
  synthetic oligonucleotide
cctctggaacctgaccacgggcatctgtgactgct

SEQ ID NO: 7 - DNA, 21 bp, Artificial Sequence
  synthetic oligonucleotide
cctctggaatctgtgactgct

SEQ ID NO: 8 - DNA, 36 bp, Artificial Sequence
  synthetic oligonucleotide
cctctggaacctcctctggaacacgggcatctgtga

SEQ ID NO: 9 - DNA, 35 bp, Artificial Sequence; identical to SEQ ID NO: 6
  synthetic oligonucleotide

SEQ ID NO: 10 - DNA, 22 bp, Artificial Sequence
  synthetic oligonucleotide
cctctgggcatctgtgactgct

SEQ ID NO: 11 - DNA, 31 bp, Artificial Sequence
  synthetic oligonucleotide
cctctggaacctacgggcatctgtgactgct

SEQ ID NO: 12 - DNA, 21 bp, Artificial Sequence
  synthetic oligonucleotide
cctctggaacctgtgactgct

SEQ ID NO: 13 - DNA, 22 bp, Artificial Sequence
  synthetic oligonucleotide
cctctgggcatctgtgactgct

SEQ ID NO: 14 - DNA, 35 bp, Artificial Sequence
  synthetic oligonucleotide
cctctggaacctgaccacgggcatctgtgactgcc

SEQ ID NO: 15 - DNA, 28 bp, Artificial Sequence
  synthetic oligonucleotide
cctctggaaccgggcatctgtgactgct

SEQ ID NO: 16 - DNA, 35 bp, Artificial Sequence
  synthetic oligonucleotide
cctctggaacctatacggccacgggcatctgtgac

SEQ ID NO: 17 - DNA, 36 bp, Artificial Sequence
  synthetic oligonucleotide
cctctggaacctggaaccacgggcatctgtgactgc

SEQ ID NO: 18 - DNA, 36 bp, Artificial Sequence
  synthetic oligonucleotide
aaacctgtccgcgtgtggcgaaggtcagggaccagt

SEQ ID NO: 19 - DNA, 36 bp, Artificial Sequence
  synthetic oligonucleotide
aaacctgtccacgtgtggcgaaggtcagggaccagt

SEQ ID NO: 20 - DNA, 30 bp, Artificial Sequence
  synthetic oligonucleotide
aaacctgtccggcgaaggtcagggaccagt

SEQ ID NO: 21 - DNA, 27 bp, Artificial Sequence
  synthetic oligonucleotide
aaacctgggcgaaggtcagggaccagt

SEQ ID NO: 22 - DNA, 31 bp, Artificial Sequence
  synthetic oligonucleotide
aaacctgtccgcaggggggtcagggaccagt

SEQ ID NO: 23 - DNA, 31 bp, Artificial Sequence
  synthetic oligonucleotide
aaacctgtaaacctgaaggtcagggaccagt

SEQ ID NO: 24 - DNA, 27 bp, Artificial Sequence
  synthetic oligonucleotide
cctctggaacgggcatctgtgactgct

SEQ ID NO: 25 - DNA, 21 bp, Artificial Sequence
  synthetic oligonucleotide
cctctggaacctgtgactgct

SEQ ID NO: 26 - DNA, 32 bp, Artificial Sequence
  synthetic oligonucleotide
cctctggaacctcacgggcatctgtgactgct

SEQ ID NO: 27 - DNA, 35 bp, Artificial Sequence; identical to SEQ ID NO: 6
  synthetic oligonucleotide

SEQ ID NO: 28 - DNA, 30 bp, Artificial Sequence; identical to SEQ ID NO: 3
  synthetic oligonucleotide

SEQ ID NO: 29 - DNA, 27 bp, Artificial Sequence
  synthetic oligonucleotide
cctcctctggaacgggcatctgtgact

SEQ ID NO: 30 - DNA, 32 bp, Artificial Sequence
  synthetic oligonucleotide
agacgtttctgaggaactgattcaggaggcca

SEQ ID NO: 31 - DNA, 28 bp, Artificial Sequence
  synthetic oligonucleotide
agacgtttctgaggaattcaggaggcca

SEQ ID NO: 32 - DNA, 24 bp, Artificial Sequence
  synthetic oligonucleotide
agacgtttctgattcaggaggcca

SEQ ID NO: 33 - DNA, 22 bp, Artificial Sequence
  synehetic oligonucleotide
agacgtttgattcaggaggcca

SEQ ID NO: 34 - DNA, 24 bp, Artificial Sequence
  synthetic oligonucleotide
agacgtttctgattcaggaggcca

SEQ ID NO: 35 - DNA, 30 bp, Artificial Sequence
  synthetic oligonucleotide
agacgtttctgaggaggattcaggaggcca

SEQ ID NO: 36 - DNA, 32 bp, Artificial Sequence
  synthetic oligonucleotide
agacgtttcgtgcaggcaactgcattcaggag

SEQ ID NO: 37 - DNA, 18 bp, Artificial Sequence
  synthetic oligonucleotide
agacgtttctggaggcca

SEQ ID NO: 38 - DNA, 32 bp, Artificial Sequence
  synthetic oligonucleotide
agtaactacctatttctgggagactatgttga

SEQ ID NO: 39 - DNA, 24 bp, Artificial Sequence
  synthetic oligonucleotide
agtaactacctatgactatgttga

SEQ ID NO: 40 - DNA, 23 bp, Artificial Sequence
  synthetic oligonucleotide
agtaactacctagactatgttga

SEQ ID NO: 41 - DNA, 23 bp, Artificial Sequence
  synthetic oligonucleotide
agtaactacctagactatgttga

SEQ ID NO: 42 - DNA, 18 bp, Artificial Sequence
  synthetic oligonucleotide
agtaactacctatgttga

SEQ ID NO: 43 - DNA, 29 bp, Artificial Sequence
  synthetic oligonucleotide
agtaactacctattttgagactatgttga

SEQ ID NO: 44 - DNA, 30 bp, Artificial Sequence
  synthetic oligonucleotide
agtaactacctatctgggagactatgttga

SEQ ID NO: 45 - DNA, 30 bp, Artificial Sequence
  synthetic oligonucleotide
agtaactacctatgtttgagactatgttga

SEQ ID NO: 46 - DNA, 32 bp, Artificial Sequence
  synthetic oligonucleotide
tacatactggagagttagttgacaaagatgaa

SEQ ID NO: 47 - DNA, 25 bp, Artificial Sequence
  synthetic oligonucleotide
tacatactggagagacaaagatgaa

SEQ ID NO: 48 - DNA, 20 bp, Artificial Sequence
  synthetic oligonucleotide
tacatactgacaaagatgaa

SEQ ID NO: 49 - DNA, 63 bp, Artificial Sequence
  synthetic oligonucleotide
gtgagcacccagcggacctcctctggaacctggaccacgggcatctgtgactgctgttctgat

SEQ ID NO: 50 - DNA, 63 bp, Artificial Sequence
  synthetic oligonucleotide
atcagaacagcagtcacagatgcccgtggtccaggttccagaggaggtccgctgggtgctcac

SEQ ID NO: 51 - DNA, 46 bp, Artificial Sequence
  synthetic oligonucleotide
acctcctctggaacctggacgatatccccacgggcatctgtgactg

SEQ ID NO: 52 - DNA, 72 bp, Artificial Sequence
  synthetic oligonucleotide
acctcctctggaacctggacataacttcgtatagcatacattatagcaatttatcacgggcatctgtgactg

SEQ ID NO: 53 - DNA, 39 bp, Artificial Sequence
  synthetic oligonucleotide
acctcctctggaacctggacccacgggcatctgtgactg

SEQ ID NO: 54 - DNA, 18 bp, Artificial Sequence
  synthetic oligonucleotide
agtcacagatgcccgtgg

SEQ ID NO: 55 - DNA, 66 bp, Artificial Sequence
  synthetic oligonucleotide
acagatgcccgtgggatatcgtaaccctgtcacagatgcagatgcccgtgggatatgtctggaacc

SEQ ID NO: 56 - DNA, 165 bp, Artificial Sequence
  synthetic oligonucleotide
ctggaaccacagatgcccgtgggatatcgtaaccctgtcacagatgcagatgcccgtgggatatgtctggaacctggacgatatcccacgggcatctgtgactgctgttctgatatgagcacttgtaagtcaccaactgaccatgggctttcagctagatggaac

SEQ ID NO: 57 - DNA, 38 bp, Artificial Sequence
  synthetic oligonucleotide
acctcctctggaacctggaccacgggcatctgtgactg

SEQ ID NO: 58 - DNA, 72 bp, Artificial Sequence; identical to SEQ ID NO: 52
  synthetic oligonucleotide

SEQ ID NO: 59 - DNA, 71 bp, Artificial Sequence
  synthetic oligonucleotide
acctcctctggaacctggacataacttcgtatagcatacattaagcaatttatcacgggcatctgtgactg

SEQ ID NO: 60 - DNA, 61 bp, Artificial Sequence
  synthetic oligonucleotide
acctcctcctatacttcgtatagcatacattatagcaatttatcacgggcatctgtgactg

SEQ ID NO: 61 - DNA, 53 bp, Artificial Sequence
  synthetic oligonucleotide
acctccgtcgtatagcatacattatagcaatttatcacgggcatctgtgactg

SEQ ID NO: 62 - DNA, 13 bp, Artificial Sequence
  synthetic oligonucleotide
catctgtgaccac

SEQ ID NO: 63 - DNA, 46 bp, Artificial Sequence
  synthetic oligonucleotide
acccagaagacgtttctgaggaactgattcaggaggccactcaaag

SEQ ID NO: 64 - DNA, 46 bp, Artificial Sequence
  synthetic oligonucleotide
ctttgagtggcctcctgaatcagttcctcagaaacgtcttctgggt

SEQ ID NO: 65 - DNA, 48 bp, Artificial Sequence
  synthetic oligonucleotide
ccaccagagagtaactacctatttctgggagactatgttgacagaggc

SEQ ID NO: 66 - DNA, 48 bp, Artificial Sequence
  synthetic oligonucleotide
gcctctgtcaacatagtctcccagaaataggtagttactctctggtgg

SEQ ID NO: 67 - DNA, 50 bp, Artificial Sequence
  synthetic oligonucleotide
ctcctcaacatacatactggagagttagttgacaaagatgaatcatttgt

SEQ ID NO: 68 - DNA, 50 bp, Artificial Sequence
  synthetic oligonucleotide
acaaatgattcatctttgtcaactaactctccagtatgtatgttgaggag

SEQ ID NO: 69 - DNA, 41 bp, Artificial Sequence
  synthetic oligonucleotide
acccagaagacgtttctgaggagtcaggaggccactcaaag

SEQ ID NO: 70 - DNA, 41 bp, Artificial Sequence
  synthetic oligonucleotide
acccagaagacgtttctgaggattcaggaggccactcaaag

SEQ ID NO: 71 - DNA, 34 bp, Artificial Sequence
  synthetic oligonucleotide
acccagaagacgtgattcaggaggccactcaaag

SEQ ID NO: 72 - DNA, 38 bp, Artificial Sequence
  synthetic oligonucleotide
acccagaagacgtttctgattcaggaggccactcaaag

SEQ ID NO: 73 - DNA, 38 bp, Artificial Sequence; identical to SEQ ID NO: 72
  synthetic oligonucleotide

SEQ ID NO: 74 - DNA, 38 bp, Artificial Sequence
  synthetic oligonucleotide
acccagaagacgcttctgattcaggaggccactcaaag

SEQ ID NO: 75 - DNA, 45 bp, Artificial Sequence
  synthetic oligonucleotide
ccaccagagagtaactacctattgggagactatgttgacagaggc

SEQ ID NO: 76 - DNA, 34 bp, Artificial Sequence
  synthetic oligonucleotide
ccaccagagagtaactacctatgttgacagaggc

SEQ ID NO: 77 - DNA, 43 bp, Artificial Sequence
  synthetic oligonucleotide
ccaccagagagtaactacctatggagactatgttgacagaggc

SEQ ID NO: 78 - DNA, 39 bp, Artificial Sequence
  synthetic oligonucleotide
ccaccagagagtaactacctagactatgttgacagaggc

SEQ ID NO: 79 - DNA, 44 bp, Artificial Sequence
  synthetic oligonucleotide
ccaccagagagtaactacctactgtagactatgttgacagaggc

SEQ ID NO: 80 - DNA, 41 bp, Artificial Sequence
  synthetic oligonucleotide
ccaccagagagtaactacctagagactatgttgacagaggc

SEQ ID NO: 81 - DNA, 47 bp, Artificial Sequence
  synthetic oligonucleotide
ctcctcaacatacatactggagagtatgacaaagatgaatcatttgt

SEQ ID NO: 82 - DNA, 50 bp, Artificial Sequence
  synthetic oligonucleotide
ctcctcaacatacatactggagagtatgtacaaaagatgaatcatttgac

SEQ ID NO: 83 - DNA, 41 bp, Artificial Sequence
  synthetic oligonucleotide
ctcctcaacatacatactggagagaaagatgaatcatttgt

SEQ ID NO: 84 - DNA, 43 bp, Artificial Sequence
  synthetic oligonucleotide
ctcctcaacatacatactggagagacaaagatgaatcatttgt

SEQ ID NO: 85 - DNA, 46 bp, Artificial Sequence
  synthetic oligonucleotide
ctcctcaacatacatactggagagatgacaaagatgaatcatttgt

SEQ ID NO: 86 - DNA, 41 bp, Artificial Sequence
  synthetic oligonucleotide
ctcctcaacatacatactggagacaaagatgaatcatttgt

SEQ ID NO: 87 - DNA, 43 bp, Artificial Sequence; identical to SEQ ID NO: 84
  synthetic oligonucleotide

SEQ ID NO: 88 - DNA, 38 bp, Artificial Sequence
  synthetic oligonucleotide
ctcctcaacatacatactgacaaagatgaatcatttgt

SEQ ID NO: 89 - DNA, 100 bp, Artificial Sequence
  synthetic oligonucleotide
gctccatgtcaaatctgcagctccacgcttcacgcctcagcaaacacagagtcagaggcagaggcagaggattgcatatctccttttctcgaaaagtaag

SEQ ID NO: 90 - DNA, 100 bp, Artificial Sequence
  synthetic oligonucleotide
cttacttttcgagaaaaggagatatgcaatcctctgcctctgcctctgactctgtgtttgctgaggcgtgaagcgtggagctgcagatttgacatggagc

SEQ ID NO: 91 - DNA, 88 bp, Artificial Sequence
  synthetic oligonucleotide
tcaaatctgcagctccacgcttcacgcataacttcgtatagcatacattatagcaatttatgcatatctccttttctcgaaaagtaag

SEQ ID NO: 92 - DNA, 104 bp, Artificial Sequence
  synthetic oligonucleotide
gctccatgtcaaatctgcagctccacgcttcacgcataacttcgtatagcatacattatagcaatttatgcatatctccttttctcgaaaagtaagcttatctc

SEQ ID NO: 93 - DNA, 108 bp, Artificial Sequence
  synthetic oligonucleotide
gctccatgtcaaatctgcagctccacgcttcacgcctcagcaaacacagagtcagaggcagaggcagaggattgcatatctccttttctcgaaaagtaagcttatctc

SEQ ID NO: 94 - DNA, 92 bp, Artificial Sequence
  synthetic oligonucleotide
gctccatgtcaaatctgcagctccacacttcgtatagcatacattatagcaatttatgcatatctccttttctcgaaaagtaagcttatctc

SEQ ID NO: 95 - DNA, 37 bp, Artificial Sequence
  synthetic oligonucleotide
agagtcagaggcagaggattgcatatctccttttctc

SEQ ID NO: 96 - DNA, 69 bp, Artificial Sequence
  synthetic oligonucleotide
gctccatgtcaaatctgcagctccacgcttcacggcatatctccttttctcgaaaagtaagcttatctc

SEQ ID NO: 97 - DNA, 109 bp, Artificial Sequence
  synthetic oligonucleotide
agatatgcataaattgctataatgtatgctatacgaagttatgcgtcttacttttcgagaaaaggagatatgtgttttcacgcaaacacagagtcagaggcagaggatt

SEQ ID NO: 98 - DNA, 88 bp, Artificial Sequence
  synthetic oligonucleotide
agctataaaaggagcataactataaaaggagcatagtctccttttctcctctgttggctttctccactgttattgttaaaattcacgc

SEQ ID NO: 99 - DNA, 73 bp, Artificial Sequence
  synthetic oligonucleotide
cttgtgtttctcataacttttgtgttttcatatacacttttactttttagcaattctcctacaggctacacgc

SEQ ID NO: 100 - DNA, 98 bp, Artificial Sequence
  synthetic oligonucleotide
gctccatgtcaaatctgcagctccacgcttcacgcataacttcgtatagcatacattatagcaatttatgcatatctccttttctcgaaacttatctc

SEQ ID NO: 101 - DNA, 63 bp, Artificial Sequence
  synthetic oligonucleotide
tacgcttcacaaacacagagtcagaggcagaggattgcatatctccttttctcgaaaagtaag

SEQ ID NO: 102 - DNA, 14 bp, Artificial Sequence
  synthetic oligonucleotide
tcgaggctacacgc

SEQ ID NO: 103 - DNA, 47 bp, Artificial Sequence
  synthetic oligonucleotide
gaaacacagagtcagaggcagaggattgcatatctccttttctcgaa

SEQ ID NO: 104 - protein, 440 aa, Artificial Sequence
  synthetic polypeptide
MASSPPKKKRKVSWKDASGWSRVDLRTLGYSQQQQEKIKPKVRSTVAQHHEALVGHGFTHAHIVALSQHPAALGTVAVTYQHIITALPEATHEDIVGVGKQWSGARALEALLTDAGELRGPPLQLDTGQLVKIAKRGGVTAMEAVHASRNALTGAPLNLTPDQVVAIASNGGGKQALESIVAQLSRPDPALAALTNDHLVALACLGGRPAMDAVKKGLPHAPELIRRVNRRIGERTSHRVASRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 105 - protein, 459 aa, Artificial Sequence
  synthetic polypeptide
MDYKDHDGDYKDHDIDYKDDDDKMAPKKKRKVGIHRGVPMVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNGGGRPALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 106 - protein, 34 aa, Artificial Sequence
  synthetic polypeptide
LTPDQVVAIASHDGGKQALETVQRLLPVLCQAHG

SEQ ID NO: 107 - protein, 34 aa, Artificial Sequence
  synthetic polypeptide
LTPDQVVAIASHDGGKQALETVQRLLPVLCQDHG

SEQ ID NO: 108 - protein, 34 aa, Artificial Sequence
  synthetic polypeptide
LTPEQVVAIASNGGGKQALETVQRLLPVLCQAHG

SEQ ID NO: 109 - protein, 34 aa, Artificial Sequence
  synthetic polypeptide
LTPDQVVAIASNGGGKQALETVQRLLPVLCQDHG

SEQ ID NO: 110 - protein, 34 aa, Artificial Sequence
  synthetic polypeptide
LTPAQVVAIASNIGGKQALETVQRLLPVLCQDHG

SEQ ID NO: 111 - protein, 34 aa, Artificial Sequence
  synthetic polypeptide
LTPDQVVAIASNIGGKQALETVQRLLPVLCQDHG

SEQ ID NO: 112 - protein, 34 aa, Artificial Sequence
  synthetic polypeptide
LTPDQVVAIANNNGGKQALETVQRLLPVLCQDHG

SEQ ID NO: 113 - protein, 34 aa, Artificial Sequence
  synthetic polypeptide
LTPDQVVAIASNNGGKQALETVQRLLPVLCQDHG

SEQ ID NO: 114 - DNA, 41 bp, Artificial Sequence
  synthetic oligonucleotide
acctcctctggaaccgatatccccacgggcatctgtgactg

SEQ ID NO: 115 - DNA, 39 bp, Artificial Sequence
  synthetic oligonucleotide
acctcctctggacgatatccccacgggcatctgtgactg

SEQ ID NO: 116 - DNA, 14 bp, Artificial Sequence
  synthetic oligonucleotide
acagatgcccgtgg

SEQ ID NO: 117 - DNA, 27 bp, Artificial Sequence
  synthetic oligonucleotide
cgtggggatatcgtccaggttccagag

SEQ ID NO: 118 - DNA, 80 bp, Artificial Sequence
  synthetic oligonucleotide
cagcaatttctactacggttgtcagtacgcaacgcacgtcgtcaggaacctggacgatatcccacgggcatctgtgactg

SEQ ID NO: 119 - DNA, 78 bp, Artificial Sequence
  synthetic oligonucleotide
cggacctcctctggaacctggacgatatcccacgggcaactctgacagttcttcggatatgagcacttgtaagtcacc

SEQ ID NO: 120 - DNA, 100 bp, Artificial Sequence
  synthetic oligonucleotide
ttctactacggttgtgagcacccagcggacctcctctggaacctggacgatatcccacgggcatctgtgactgctgttctgatatgagcacttgtaagtc

SEQ ID NO: 121 - DNA, 72 bp, Artificial Sequence; identical to SEQ ID NO: 52
  synthetic oligonucleotide

SEQ ID NO: 122 - DNA, 107 bp, Artificial Sequence
  synthetic oligonucleotide
cagcaatttctactacggttgtcagtacgcaacgcacgtcgtcaggaacctggacataacttcgtatagcatacattatagcaatttatcacgggcatctgtgactg

SEQ ID NO: 123 - DNA, 105 bp, Artificial Sequence
  synthetic oligonucleotide
cggacctcctctggaacctggacataacttcgtatagcatacattatagcaatttatcacgggcaactctgacagttcttcggatatgagcacttgtaagtcacc

SEQ ID NO: 124 - DNA, 24 bp, Danio rerio
gtgagcacccagcggacctcctct

SEQ ID NO: 125 - DNA, 21 bp, Danio rerio
atcagaacaacagtcagagat

SEQ ID NO: 126 - DNA, 18 bp, Danio rerio
ggaacctggaccacgggc

SEQ ID NO: 127 - DNA, 24 bp, Danio rerio
tgcaacactgagctctgtaaacct

SEQ ID NO: 128 - DNA, 24 bp, Danio rerio
ctgctgccgactggaccctgaggt

SEQ ID NO: 129 - DNA, 15 bp, Danio rerio
gtccgcgtgtggcga

SEQ ID NO: 130 - DNA, 15 bp, Danio rerio
acccagaagacgttt

SEQ ID NO: 131 - DNA, 16 bp, Danio rerio
ctttgagtggcctcct

SEQ ID NO: 132 - DNA, 15 bp, Danio rerio
ctgaggaactgattc

SEQ ID NO: 133 - DNA, 18 bp, Danio rerio
ctcctcaacatacatact

SEQ ID NO: 134 - DNA, 17 bp, Danio rerio
gcctctgtcaacatagt

SEQ ID NO: 135 - DNA, 14 bp, Danio rerio
cctatttctgggag

SEQ ID NO: 136 - DNA, 18 bp, Danio rerio
ctcctcaacatacatact

SEQ ID NO: 137 - DNA, 16 bp, Danio rerio
acaaatgattcatctt

SEQ ID NO: 138 - DNA, 16 bp, Danio rerio
ggagagttagttgaca

SEQ ID NO: 139 - DNA, 23 bp, Danio rerio
gtcaaatctgcagctccacgctt

SEQ ID NO: 140 - DNA, 19 bp, Danio rerio
cctctgcctctgactctgt

SEQ ID NO: 141 - DNA, 15 bp, Danio rerio
cacgcctcagcaaac

SEQ ID NO: 142 - DNA, 33 bp, Artificial Sequence
  synthetic oligonucleotide
gttcacacaaaatgtctctcaagtctctaaatc

SEQ ID NO: 143 - DNA, 25 bp, Artificial Sequence
  synthetic oligonucleotide
agtggccagtgagtgtatgttacct

SEQ ID NO: 144 - DNA, 25 bp, Artificial Sequence
  synthetic oligonucleotide
cgtgaaagagacagcgaagggattg

SEQ ID NO: 145 - DNA, 29 bp, Artificial Sequence
  synthetic oligonucleotide
agaaactaccattgtcacactgagcgaag

SEQ ID NO: 146 - DNA, 18 bp, Artificial Sequence
  synthetic oligonucleotide
gttacggctcaagacgtc

SEQ ID NO: 147 - DNA, 18 bp, Artificial Sequence
  synthetic oligonucleotide
caggatgccctctttaac

SEQ ID NO: 148 - DNA, 18 bp, Artificial Sequence
  synthetic oligonucleotide
gatgttcatggtcagtac

SEQ ID NO: 149 - DNA, 20 bp, Artificial Sequence
  synthetic oligonucleotide
tgattgaggcacattcatgg

SEQ ID NO: 150 - DNA, 20 bp, Artificial Sequence
  synthetic oligonucleotide
ttgttgtccttgcaaagctg

SEQ ID NO: 151 - DNA, 18 bp, Artificial Sequence
  synthetic oligonucleotide
tctagaggattcgctgat

SEQ ID NO: 152 - DNA, 28 bp, Artificial Sequence
  synthetic oligonucleotide
ccctgattgtggaacttttcagaacgta

SEQ ID NO: 153 - DNA, 27 bp, Artificial Sequence
  synthetic oligonucleotide
tggtttggaattagtgcagcatgagta

SEQ ID NO: 154 - DNA, 33 bp, Artificial Sequence
  synthetic oligonucleotide
taacttcgtatagcatacattatagcaatttat

SEQ ID NO: 155 - DNA, 31 bp, Artificial Sequence
  synthetic oligonucleotide
ataaattgctataatgtatgctatacgaagt

SEQ ID NO: 156 - DNA, 31 bp, Artificial Sequence
  synthetic oligonucleotide
acttcgtatagcatacattatagcaatttat